(19) United States
(12) Patent Application Publication
(10) Pub. No.: US 2019/0092916 A1
(43) Pub. Date: Mar. 28, 2019

(54) PLASMA TREATMENT WITH NON-POLYMERIZING COMPOUNDS THAT LEADS TO REDUCED BIOMOLECULE ADHESION TO THERMOPLASTIC ARTICLES

(71) Applicant: SIO2 MEDICAL PRODUCTS, INC., Auburn, AL (US)

(72) Inventors: Ahmad Taha, Auburn, AL (US); John T. Felts, Alameda, CA (US)

(21) Appl. No.: 15/536,811

(22) PCT Filed: Dec. 16, 2015
(86) PCT No.: PCT/US2015/066169

§ 371 Date: Jun. 16, 2017

Related U.S. Application Data
(60) Provisional application No. 62/093,194, filed on Dec. 17, 2014.
(60) Provisional application No. 62/114,494, filed on Feb. 10, 2015.
(60) Provisional application No. 62/155,090, filed on Apr. 30, 2015.
(60) Provisional application No. 62/243,392, filed on Oct. 19, 2015.

(51) Int. Cl. C08J 7/12; C12M 1/00

(52) U.S. Cl. CPC C08J 7/123; C08J 2323/12; C12M 23/20

(57) ABSTRACT

A method is provided for treating a surface of a substrate. The method includes treating the surface with plasma comprising one or more non-polymerizing compounds. The converted surface has a biomolecule recovery percentage greater than the biomolecule recovery percentage of the surface prior to treatment according to the method.

### PRIORITY CLAIMS

This specification claims the priority of U.S. Ser. Nos. 62/093,194, filed Dec. 17, 2014; 62/114,494, filed Feb. 10, 2015; 62/155,090, filed Apr. 30, 2015; and 62/243,392, filed Oct. 19, 2015. Each of these four specifications and their drawings are incorporated here by reference in their entirety to provide continuity of disclosure.

### FIELD OF INVENTION

The invention relates generally to treating a surface, or surface modification of a plastic substrate, to reduce biomolecule adhesion to the surface. More particularly, the invention relates to plasma treatment or surface modification of a plastic substrate, e.g., a medical device or item of laboratory ware, using non-polymerizing compounds to reduce protein adhesion to the substrate surface.

### BACKGROUND

In blood, biomolecule, and blood analyte testing, it is desirable to minimize biomolecule adsorption and binding to plastic ware used with these biological substances. Plastic microwell plates, chromatography vials, and other containers, as well as pipettes (sometimes spelled “pipets”), pipette tips, centrifuge tubes, microscope slides, and other types of laboratory ware (also known as labware) used to prepare and transfer samples commonly have hydrophobic surfaces and readily adsorb biomolecules such as proteins, DNA, and RNA. Surfaces of these and other types of laboratory ware components made of polymeric plastic can cause binding of the biomolecule samples. It is thus a desire to provide surfaces for plastic laboratory ware and other articles that contact biological substances, to reduce a wide range of biomolecules from adhering.

### SUMMARY

Accordingly, in one aspect, the invention is directed to a method for treating a surface, optionally a surface of a substrate or a surface of a material. The method is carried out by treating the surface with conversion plasma of one or more non-polymerizing compounds, conditioning plasma of one or more non-polymerizing compounds, or both, to form a converted surface.

In a first more detailed embodiment, the invention is directed to a method for treating a surface of a substrate. The method includes at least two treatment steps. The first treatment step includes treating the surface with remote conditioning plasma of one or more non-polymerizing compounds. The second treatment step includes treating the surface with remote conversion plasma of water to form a converted surface. The converted surface has a biomolecule recovery percentage greater than the biomolecule recovery percentage of the surface prior to treatment according to the method.

In a second more detailed embodiment, the invention is directed to a method for treating a surface of a material. The method is carried out by treating the surface with conversion plasma of water; a volatile, polar, organic compound; a C₁-C₁₂ hydrocarbon and oxygen; a C₁-C₁₂ hydrocarbon and nitrogen; a silicon-containing gas; or a combination of two or more of these. The result is to form a converted surface.

### BRIEF DESCRIPTION OF THE DRAWINGS

The invention will be described in conjunction with the following drawings in which like reference numerals designate like elements and wherein:

FIG. 1 illustrates generically described remote conversion plasma treatment apparatus useful in the first embodiment, certain features of which are optional.

FIG. 2 illustrates an exemplary plasma reactor configuration for carrying out remote conversion plasma treatment of microplates according to the first more detailed embodiment.

FIG. 3 is a bar graph illustrating comparative biomolecule recovery results between untreated polypropylene microplates, Eppendorf brand microplates and microplates treated with an exemplary remote conversion plasma treatment process according to the first more detailed embodiment.

FIG. 4 is a bar graph illustrating comparative biomolecule recovery results between microplates treated with an exemplary remote conversion plasma treatment process according to the first more detailed embodiment and microplates treated with the same process steps and conditions except using direct conversion plasma treatment instead of remote conversion plasma treatment.

FIG. 5 is a bar graph illustrating comparative biomolecule recovery results between microplates treated with an exemplary remote conversion plasma treatment process according to the first more detailed embodiment and microplates treated with only the non-polymerizing compound step and without the second step.

FIG. 6 illustrates an exemplary radio-frequency-excited plasma reactor configuration according to FIG. 1 for carrying out remote conversion plasma treatment of microplates according to the first more detailed embodiment.

FIG. 7 illustrates another exemplary plasma reactor configuration according to FIG. 1 for carrying out remote conversion plasma treatment of microplates according to the first more detailed embodiment.

FIG. 8 is an exemplary microwave-excited plasma reactor configuration according to FIG. 1 for carrying out remote conversion plasma treatment of microplates according to the first more detailed embodiment.

FIG. 9 is a plot of biomolecule (TFN) recovery from untreated polypropylene beakers, treated polypropylene beakers according to the first more detailed embodiment, and glass beakers.

FIG. 10 shows an exemplary reactor configuration for carrying out either the first embodiment or the second embodiment of the present process. Another suitable reactor configuration is that of FIG. 2 as shown and described in U.S. Pat. No. 7,985,188, incorporated here by reference.

FIG. 11 is a plot of protein recovery versus concentration of protein (BSA) for Example 6.

FIG. 12 is a plot of protein recovery versus concentration of protein (PrA) for Example 6.

FIG. 13 is a plot of protein recovery versus concentration of protein (PrG) for Example 6.

The following reference characters are used in this description and the accompanying Figures:

| | |
|---|---|
| 9 | Apparatus of the first more detailed embodiment |
| 10 | Treatment volume |
| 11 | Reaction chamber wall (optional) |
| 12 | Fluid source |
| 13 | Fluid inlet |
| 14 | Substrate |
| 15 | Plasma zone |
| 16 | Shield (optional) |
| 17 | Treatment gas |
| 18 | Plasma energy source |
| 19 | Lid (optional) |
| 20 | Plasma (boundary) |
| 21 | Substrate support (optional) |
| 22 | Vacuum source (optional) |
| 23 | Applicator |
| 24 | Remote conversion plasma |
| 26 | Flat spot (of 20) (optional) |
| 28 | Front surface (of 14) |
| 30 | Back surface (of 14) |
| 32 | Well (of 14) (optional) |
| 34 | Untreated polypropylene plot |
| 36 | Treated polypropylene plot |
| 38 | Glass plot |
| 110 | Chamber |
| 112 | Bottom (of 110) |
| 114 | Lid (of 110) |
| 116 | Vacuum conduit |
| 118 | Vacuum pump |
| 120 | Valve |
| 122 | Gas inlet |
| 124 | Processing area |
| 126 | Gas system |
| 128 | Mass flow controller |
| 130 | Compressed gas source |
| 132 | Capillary |
| 134 | Manifold |
| 136 | Shut-off valve |
| 138 | Electrode |
| 140 | Matching network |
| 142 | RF power supply |
| 144 | Coaxial cable |

### DETAILED DESCRIPTION OF THE PREFERRED EMBODIMENTS

According to the invention, methods are disclosed for reducing biomolecule adhesion to a surface. A method for treating a surface, optionally an entire or partial surface of a substrate or a surface of a material, is provided, most generally comprising treating the surface with conversion plasma of one or more non-polymerizing compounds to form a converted surface.

The term “biomolecule” is used respecting any embodiment to include any nucleotides or peptides, or any combination of them. Nucleotides include oligonucleotides and polynucleotides, also known as nucleic acids, for example deoxyribonucleic acid (DNA) and ribonucleic acid (RNA). Peptides include amino acids, oligopeptides, polypeptides, and proteins. Nucleotides and peptides further include modified or derivatized nucleotides and peptides that adhere to a surface that is not treated according to the present invention.

The presently defined biomolecules include but are not limited to one or more of the following aqueous proteins: mammal serum albumin, for example Bovine Serum Albumin (BSA); Fibrinogen (FBG); Transferrin (TFN), for example blood serotransferrin (or siderophilin, also known as transferrin); lactotransferrin (lactoferrin); milk transferrin; egg white ovotransferrin (conalbumin); membrane-associated melanotransferrin; Protein A (PrA); Protein G (PrG); Protein A/G; Protein L; Insulin, for example hexameric insulin, monomeric insulin, porcine insulin, human insulin, recombinant insulin and pharmaceutical grades of insulin; pharmaceutical protein; blood or blood component proteins; or any recombinant form, modification, full length precursor, signal peptide, pro-peptide, or mature variant of these proteins; and a combination of two or more of these.

Biomolecule adhesion to a surface is defined for any embodiment as a reduction of the aqueous concentration of a biomolecule dispersed in an aqueous medium stored in contact with the surface. It is not limited by the mechanism of reduction of concentration, whether literally “adhesion,” adsorption, or another mechanism.

“Plasma,” as referenced in any embodiment, has its conventional meaning in physics of one of the four fundamental states of matter, characterized by extensive ionization of its constituent particles, a generally gaseous form, and incandescence (i.e. it produces a glow discharge, meaning that it emits light).

“Conversion plasma treatment” refers to any plasma treatment that reduces the adhesion of one or more biomolecules to a treated surface.

“Conditioning plasma treatment” refers to any plasma treatment of a surface to prepare the surface for further conversion plasma treatment. “Conditioning plasma treatment” includes a plasma treatment that, in itself, reduces the adhesion of one or more biomolecules to a treated surface, but is followed by conversion plasma treatment that further reduces the adhesion of one or more biomolecules to a treated surface. “Conditioning plasma treatment” also includes a plasma treatment that, in itself, does not reduce the adhesion of one or more biomolecules to a treated surface.

A “remote” conversion plasma treatment, generally speaking, is conversion plasma treatment of a surface located at a “remote” point where the radiant energy density of the plasma, for example in Joules per cm³, is substantially less than the maximum radiant energy density at any point of the plasma glow discharge (referred to below as the “brightest point”), but the remote surface is close enough to some part of the glow discharge to reduce the adhesion of one or more biomolecules to the treated remote surface. “Remote” is defined in the same manner respecting a remote conditioning plasma treatment, except that the remote surface must be close enough to some part of the glow discharge to condition the surface.

The radiant energy density at the brightest point of the plasma is determined spectrophotometrically by measuring the radiant intensity of the most intense emission line of light in the visible spectrum (380 nanometer (nm) to 750 nm wavelength) at the brightest point. The radiant energy density at the remote point is determined spectrophotometrically by measuring the radiant energy density of the same emission line of light at the remote point. “Remoteness” of a point is quantified by measuring the ratio of the radiant energy density at the remote point to the radiant energy density at the brightest point. The present specification and claims define “remote” quantitatively as a specific range of that ratio. Broadly, the ratio is from 0 to 0.5, alternatively from 0 to 0.25, alternatively about 0, alternatively exactly 0. Remote conversion plasma treatment can be carried out where the ratio is zero, even though that indicates no measurable visible light at the remote point, because the dark discharge region or afterglow region of plasma contain energetic species that, although not energetic enough to emit light, are energetic enough to modify the treated surface to reduce the adhesion of one or more biomolecules.

A “non-polymerizing compound” is defined operationally for all embodiments as a compound that does not polymerize on a treated surface or otherwise form an additive coating under the conditions used in a particular plasma treatment of the surface. Numerous, non-limiting examples of compounds that can be used under non-polymerizing conditions are the following: O₂, N₂, air, O₃, N₂O, H₂, H₂O₂, NH₃, Ar, He, Ne, and combinations of any of two or more of the foregoing. These may also include alcohols, organic acids, and polar organic solvents as well as materials that can be polymerized under different plasma conditions from those employed. “Non-polymerizing” includes compounds that react with and bond to a preexisting polymeric surface and locally modify its composition at the surface. The essential characterizing feature of a non-polymerizing coating is that it does not build up thickness (i.e. build up an additive coating) as the treatment time is increased.

A “substrate” is an article or other solid form (such as a granule, bead, or particle).

A “surface” is broadly defined as either an original surface (a “surface” also includes a portion of a surface wherever used in this specification) of a substrate, or a coated or treated surface prepared by any suitable coating or treating method, such as liquid application, condensation from a gas, or chemical vapor deposition, including plasma enhanced chemical vapor deposition carried out under conditions effective to form a coating on the substrate.

A converted surface is defined for all embodiments as a surface that has been plasma treated as described in this specification, and that exhibits reduced adhesion by biomolecules as a result of such treatment.

The “material” in any embodiment can be any material of which a substrate is formed, including but not limited to a thermoplastic material, optionally a thermoplastic injection moldable material. The substrate according to any embodiment may be made, for example, from material including, but not limited to: an olefin polymer; polypropylene (PP); polyethylene (PE); cyclic olefin copolymer (COC); cyclic olefin polymer (COP); polymethylpentene; polyester; polyethylene terephthalate; polyethylene naphthalate; polybutylene terephthalate (PBT); PVdC (polyvinylidene chloride); polyvinyl chloride (PVC); polycarbonate; polymethylmethacrylate; polylactic acid; polylactic acid; polystyrene; hydrogenated polystyrene; poly(cyclohexylethylene) (PCHE); epoxy resin; nylon; polyurethane polyacrylonitrile; polyacrylonitrile (PAN); an ionomeric resin; or Surlyn® ionomeric resin.

The term “vessel” as used throughout this specification may be any type of article that is adapted to contain or convey a liquid, a gas, a solid, or any two or more of these. One example of a vessel is an article with at least one opening (e.g., one, two or more, depending on the application) and a wall defining an interior contacting surface.

The present method for treating a surface, optionally a surface of a substrate, includes treating the surface with conversion plasma of one or more non-polymerizing compounds, in a chamber, to form a converted surface.

A wide variety of different surfaces can be treated according to any embodiment. One example of a surface is a vessel lumen surface, where the vessel is, for example, a vial, a bottle, a jar, a syringe, a cartridge, a blister package, or an ampoule. For more examples, the surface of the material can be a fluid contact surface of an article of labware, for example a microplate, a centrifuge tube, a pipette tip, a well plate, a microwell plate, an ELISA plate, a microtiter plate, a 96-well plate, a 384-well plate, a centrifuge tube, a chromatography vial, an evacuated blood collection tube, or a specimen tube.

The treated surface of any embodiment can be a coating or layer of PECVD deposited SiOₓC_{y}H_{z} or SiNₓC_{y}H_{z}, in which x is from about 0.5 to about 2.4 as measured by X-ray photoelectron spectroscopy (XPS), y is from about 0.6 to about 3 as measured by XPS, and z is from about 2 to about 9 as measured by Rutherford backscattering spectrometry (RBS). Another example of the surface to be treated is a barrier coating or layer of SiOₓ, in which x is from about 1.5 to about 2.9 as measured by XPS, alternatively an oxide or nitride of an organometallic precursor that is a compound of a metal element from Group III and/or Group IV of the Periodic Table, e.g. in Group III: Boron, Aluminum, Gallium, Indium, Thallium, Scandium, Yttrium, or Lanthanum, (Aluminum and Boron being preferred), and in Group IV: Silicon, Germanium, Tin, Lead, Titanium, Zirconium, Hafnium, or Thorium (Silicon and Tin being preferred).

The gas or gases employed to treat the surface in any embodiment can be an inert gas or a reactive gas, and can be any of the following: O₂, N₂, air, O₃, N₂O, NO₂, N₂O₄, H₂, H₂O₂, H₂O, NH₃, Ar, He, Ne, Xe, Kr, a nitrogen-containing gas, other non-polymerizing gases, gas combinations including an Ar/O₂ mix, an N₂/O₂ mix following a pre-treatment conditioning step with Ar, a volatile and polar organic compound, the combination of a C₁-C₁₂ hydrocarbon and oxygen; the combination of a C₁-C₁₂ hydrocarbon and nitrogen; a silicon-containing gas; or a combination of two or more of these. The treatment employs a non-polymerizing gas as defined in this specification.

The volatile and polar organic compound of any embodiment can be, for example water, for example tap water, distilled water, or deionized water; an alcohol, for example a C₁-C₁₂ alcohol, methanol, ethanol, n-propanol, isopropanol, n-butanol, isobutanol, s-butanol, t-butanol; a glycol, for example ethylene glycol, propylene glycol, butylene glycol, polyethylene glycol, and others; glycerine, a C₁-C₁₂ linear or cyclic ether, for example dimethyl ether, diethyl ether, dipropyl ether, dibutyl ether, glyme (CH₃OCH₂CH₂OCH₃); cyclic ethers of formula —CH₂CH₂Oₙ— such as diethylene oxide, triethylene oxide, and tetraethylene oxide; cyclic amines; cyclic esters (lactones), for example acetolactone, propiolactone, butyrolactone, valerolactone, and caprolactone; a C₁-C₁₂ aldehyde, for example formaldehyde, acetaldehyde, propionaldehyde, or butyraldehyde; a C₁-C₁₂ ketone, for example acetone, diethylketone, dipropylketone, or dibutylketone; a C₁-C₁₂ carboxylic acid, for example formic acid, acetic acid, propionic acid, or butyric acid; ammonia, a C₁-C₁₂ amine, for example methylamine, dimethylamine, ethylamine, diethylamine, propylamine, butylamine, pentylamine, hexylamine, heptylamine, octylamine, nonylamine, decylamine, undecylamine, or dodecylamine; hydrogen fluoride, hydrogen chloride, a C₁-C₁₂ epoxide, for example ethylene oxide or propylene oxide; or a combination of any two or more of these.

The C₁-C₁₂ hydrocarbon of any embodiment optionally can be methane, ethane, ethylene, acetylene, n-propane, i-propane, propene, propyne; n-butane, i-butane, t-butane, butane, 1-butyne, 2-butyne, or a combination of any two or more of these.

The silicon-containing gas of any embodiment can be a silane, an organosilicon precursor, or a combination of any two or more of these. The silicon-containing gas can be an acyclic or cyclic, substituted or unsubstituted silane, optionally comprising, consisting essentially of, or consisting of any one or more of: Si₁-Si₄ substituted or unsubstituted silanes, for example silane, disilane, trisilane, or tetrasilane; hydrocarbon or halogen substituted Si₁-Si₄ silanes, for example tetramethylsilane (TetraMS), tetraethyl silane, tetrapropylsilane, tetrabutylsilane, trimethylsilane (TriMS), triethyl silane, tripropylsilane, tributylsilane, trimethoxysilane, a fluorinated silane such as hexafluorodisilane, a cyclic silane such as octamethylcyclotetrasilane or tetramethylcyclotetrasilane, or a combination of any two or more of these. The silicon-containing gas can be a linear siloxane, a monocyclic siloxane, a polycyclic siloxane, a polysilsesquioxane, an alkyl trimethoxysilane, a linear silazane, a monocyclic silazane, a polycyclic silazane, a polysilsesquiazane, or a combination of any two or more of these, for example hexamethyldisiloxane (HMDSO), tetramethyldisiloxane (TMDSO), octamethylcyclotetrasiloxane (OMCTS), tetramethyldisilazane, hexamethyldisilazane, octamethyltrisilazane, octamethylcyclotetrasilazane, tetramethylcyclotetrasilazane, or a combination of any two or more of these.

The electrical power used to excite the plasma used in plasma treatment in any embodiment, can be, for example, from 1 to 1000 Watts, optionally from 100 to 900 Watts, optionally from 50 to 600 Watts, alternatively 100 to 500 Watts, optionally from 500 to 700 Watts, optionally from 1 to 100 Watts, optionally from 1 to 30 Watts, optionally from 1 to 10 Watts, optionally from 1 to 5 Watts.

The frequency of the electrical power used to excite the plasma used in plasma treatment, in any embodiment, can be any type of energy that will ignite plasma in the plasma zone. For example, it can be direct current (DC) or alternating current (electromagnetic energy) having a frequency from 3 Hz to 300 GHz. Electromagnetic energy in this range generally includes radio frequency (RF) energy and microwave energy, more particularly characterized as extremely low frequency (ELF) of 3 to 30 Hz, super low frequency (SLF) of 30 to 300 Hz, voice or ultra-low frequency (VF or ULF) of 300 Hz to 3 kHz, very low frequency (VLF) of 3 to 30 kHz, low frequency (LF) of 30 to 300 kHz, medium frequency (MF) of 300 kHz to 3 MHz, high frequency (HF) of 3 to 30 MHz, very high frequency (VHF) of 30 to 300 MHz, ultra-high frequency (UHF) of 300 MHz to 3 GHz, super high frequency (SHF) of 3 to 30 GHz, extremely high frequency (EHF) of 30 to 300 GHz, or any combination of two or more of these frequencies. For example, high frequency energy, commonly 13.56 MHz, is useful RF energy, and ultra-high frequency energy, commonly 2.54 GHz, is useful microwave energy, as two non-limiting examples of commonly used frequencies.

The plasma exciting energy, in any embodiment, can either be continuous during a treatment step or pulsed multiple times during the treatment step. If pulsed, it can alternately pulse on for times ranging from one millisecond to one second, and then off for times ranging from one millisecond to one second, in a regular or varying sequence during plasma treatment. One complete duty cycle (one “on” period plus one “off” period) can be 1 to 2000 milliseconds (ms), alternatively 1 to 1000 milliseconds (ms), alternatively 2 to 500 ms, alternatively 5 to 100 ms, alternatively 10 to 100 ms long.

Optionally in any embodiment, the relation between the power on and power off portions of the duty cycle can be, for example, power on 1-90 percent of the time, alternatively on 1-80 percent of the time, alternatively on 1-70 percent of the time, alternatively on 1-60 percent of the time, alternatively on 1-50 percent of the time, alternatively on 1-45 percent of the time, alternatively on 1-40 percent of the time, alternatively on 1-35 percent of the time, alternatively on 1-30 percent of the time, alternatively on 1-25 percent of the time, alternatively on 1-20 percent of the time, alternatively on 1-15 percent of the time, alternatively on 1-10 percent of the time, alternatively on 1-5 percent of the time, and power off for the remaining time of each duty cycle.

The plasma pulsing described in Mark J. Kushner, Pulsed Plasma-Pulsed Injection Sources For Remote Plasma Activated Chemical Vapor Deposition, J. APPL. PHYS. 73, 4098 (1993), can optionally be used.

The flow rate of process gas during plasma treatment according to any embodiment can be from 1 to 300 sccm (standard cubic centimeters per minute), alternatively 1 to 200 sccm, alternatively from 1 to 100 sccm, alternatively 1-50 sccm, alternatively 5-50 sccm, alternatively 1-10 sccm.

Optionally in any embodiment, the plasma chamber is reduced to a base pressure from 0.001 milliTorr (mTorr, 0.00013 Pascal) to 100 Torr (13,000 Pascal) before feeding gases. Optionally the feed gas pressure in any embodiment can range from 0.001 to 10,000 mTorr (0.00013 to 1300 Pascal), alternatively from 1 mTorr to 10 Torr (0.13 to 1300 Pascal), alternatively from 0.001 to 5000 mTorr (0.00013 to 670 Pascal), alternatively from 1 to 1000 milliTorr (0.13 to 130 Pascal).

The treatment volume in which the plasma is generated in any embodiment can be, for example, from 100 mL to 50 liters, preferably 8 liters to 20 liters.

The plasma treatment time in any embodiment can be, for example, from 1 to 300 seconds, alternatively 3 to 300 sec., alternatively 30 to 300 sec., alternatively 150 to 250 sec., alternatively 150 to 200 sec., alternatively from 90 to 180 seconds.

The number of plasma treatment steps can vary, in any embodiment. For example one plasma treatment can be used; alternatively two or more plasma treatments can be used, employing the same or different conditions.

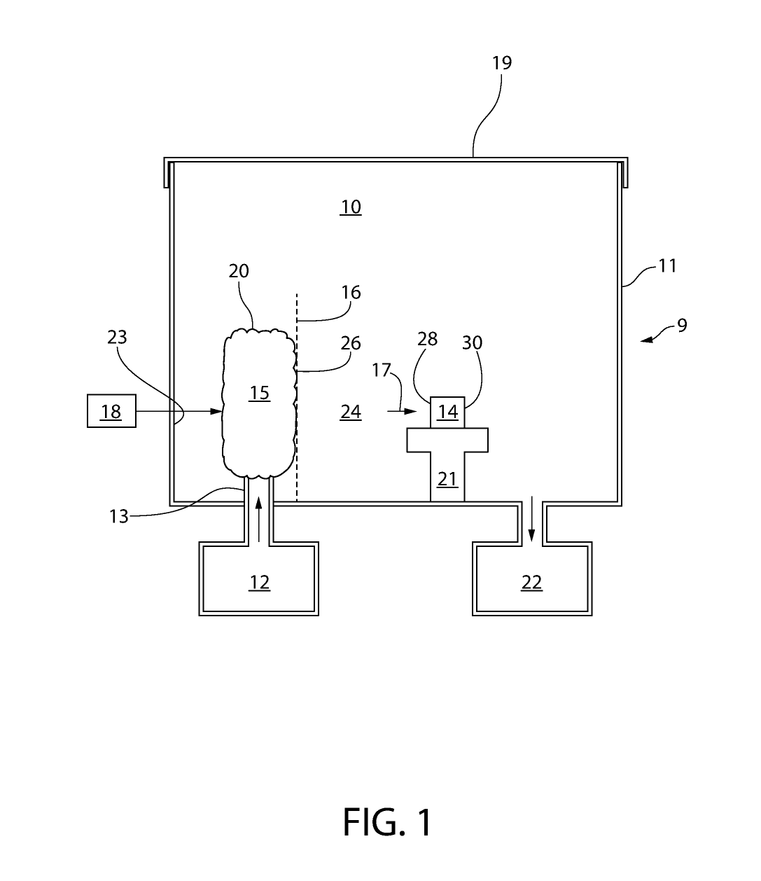

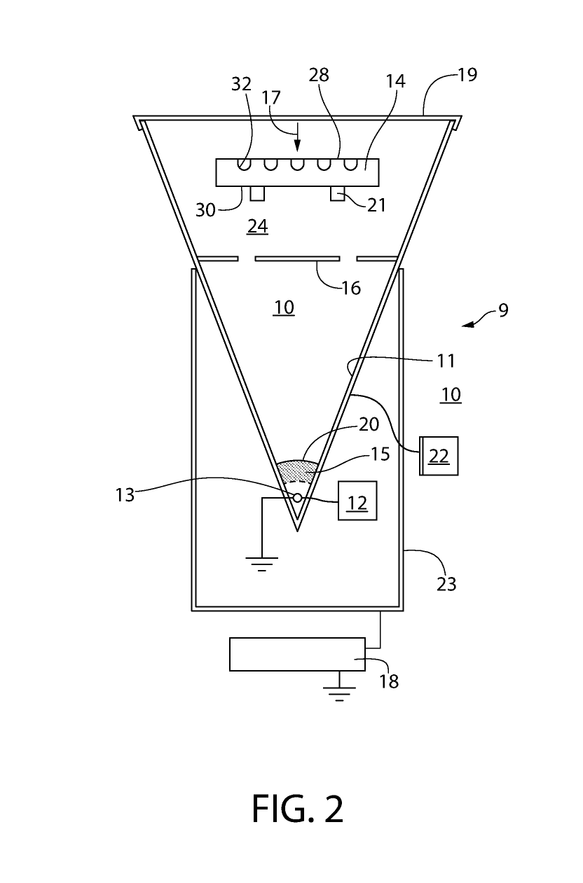

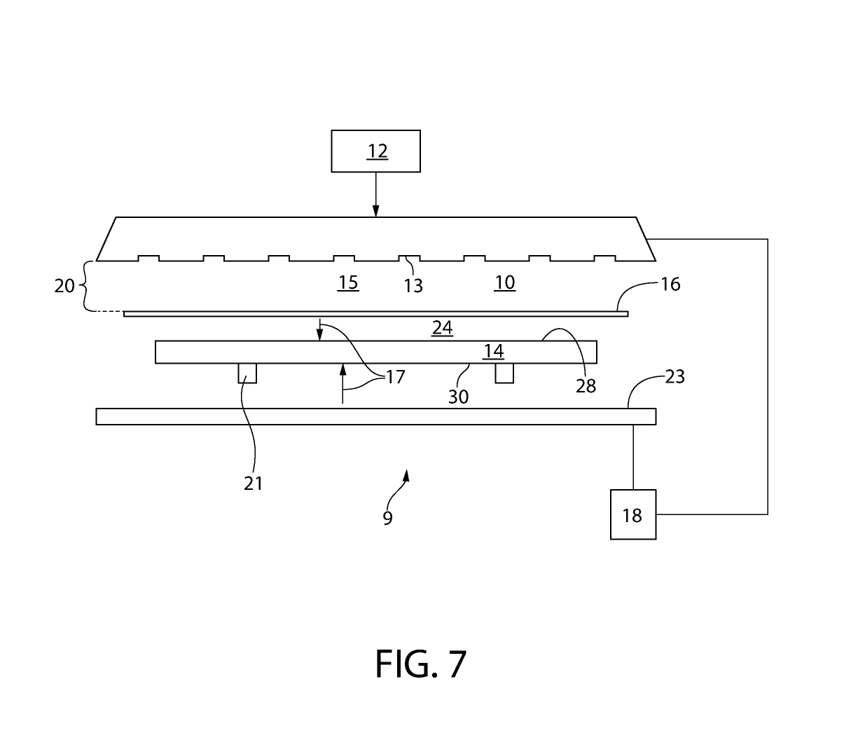

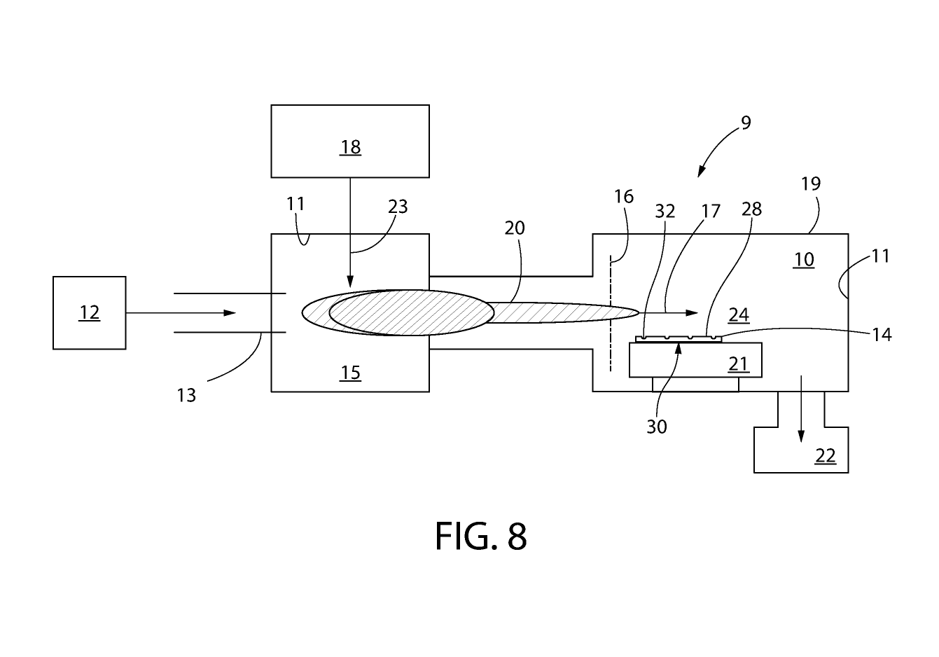

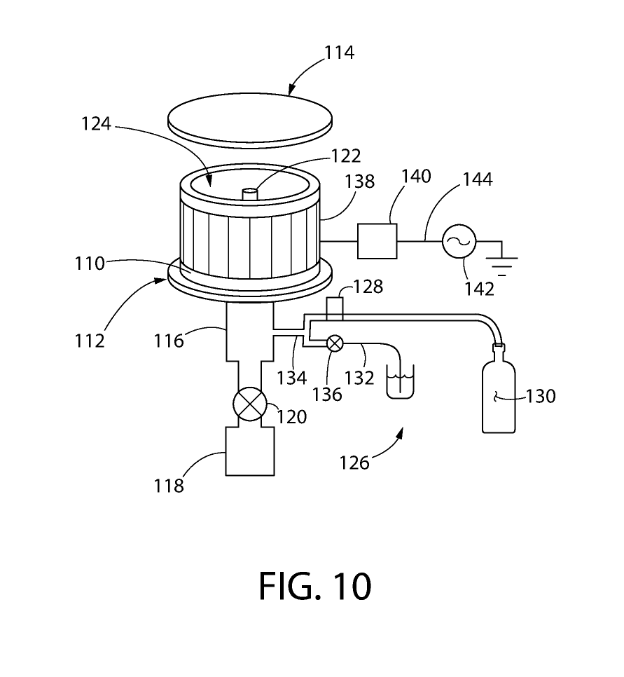

In any embodiment, the plasma treatment apparatus employed can be any suitable apparatus, for example that of FIG. 1, FIG. 7, FIG. 8, or FIG. 10 described in this specification, as several examples. Plasma treatment apparatus of the type that employs the lumen of the vessel to be treated as a vacuum chamber, shown for example in U.S. Pat. No. 7,985,188, FIG. 2, can also be used in any embodiment.

The plasma treatment process of any embodiment optionally can be combined with treatment using an ionized gas. The ionized gas can be, as some examples, any of the gases identified as suitable for plasma treatment. The ionized gas can be delivered in any suitable manner. For example, it can be delivered from an ionizing blow-off gun or other ionized gas source. A convenient gas delivery pressure is from 1-120 psi (pounds per square inch) (6 to 830 kPa, kiloPascals) (gauge or, alternatively, absolute pressure), optionally 50 psi (350 kPa). The water content of the ionized gas can be from 0 to 100%. The polar-treated surface with ionized gas can be carried out for any suitable treatment time, for example from 1-300 seconds, optionally for 10 seconds.

After the plasma treatment(s) of any embodiment, the treated surface, for example a vessel lumen surface, can be contacted with an aqueous protein. Some non-limiting examples of suitable proteins are the aqueous protein comprises: mammal serum albumin, for example Bovine Serum Albumin (BSA); Fibrinogen (FBG); Transferrin (TFN), for example blood serotransferrin (or siderophilin, also known as transferrin); lactotransferrin (lactoferrin); milk transferrin; egg white ovotransferrin (conalbumin); and membrane-associated melanotransferrin; Protein A (PrA); Protein G (PrG); Protein A/G; Protein L; Insulin, for example hexameric insulin, monomeric insulin, porcine insulin, human insulin, recombinant insulin and pharmaceutical grades of insulin; Pharmaceutical protein; blood or blood component proteins; or any recombinant form, modification, full length precursor, signal peptide, propeptide, or mature variant of these proteins; or a combination of two or more of these.

Optionally, the converted surface has a protein recovery percentage greater than the protein recovery percentage of the untreated surface for at least one of Bovine Serum Albumin (BSA); Fibrinogen (FBG); Transferrin (TFN), for example blood serotransferrin (or siderophilin, also known as transferrin); lactotransferrin (lactoferrin); milk transferrin; egg white ovotransferrin (conalbumin); and membrane-associated melanotransferrin; Protein A (PrA); Protein G (PrG); Protein A/G; Protein L; Insulin, for example hexameric insulin, monomeric insulin, porcine insulin, human insulin, recombinant insulin and pharmaceutical grades of insulin; pharmaceutical protein; blood or blood component proteins; or any recombinant form, modification, full length precursor, signal peptide, propeptide, or mature variant of these proteins.

### First More Detailed Embodiment

A vessel having a substrate according to the first more detailed embodiment may be made, for example, from any of the materials defined above. For applications in which clear and glass-like polymers are desired (e.g., for syringes and vials), a cyclic olefin polymer (COP), cyclic olefin copolymer (COC), polymethylmethacrylate, polyethylene terephthalate or polycarbonate may be preferred. Such substrates may be manufactured, e.g., by injection molding or injection stretch blow molding (which is also classified as injection molding in any embodiment of this disclosure), to very tight and precise tolerances (generally much tighter than achievable with glass). Plasma treated glass substrates, for example borosilicate glass substrates, are also contemplated.

A vessel according to the first more detailed embodiment can be a sample tube, e.g. for collecting or storing biological fluids like blood or urine, a syringe (or a part thereof, for example a syringe barrel) for storing or delivering a biologically active compound or composition, e.g., a medicament or pharmaceutical composition, a vial for storing biological materials or biologically active compounds or compositions, a pipe, e.g., a catheter for transporting biological materials or biologically active compounds or compositions, or a cuvette for holding fluids, e.g., for holding biological materials or biologically active compounds or compositions. Other non-limiting examples of contemplated vessels include well or non-well slides or plates, for example titer plates or microtiter plates (a.k.a. microplates). Other examples of vessels include measuring and delivery devices such as pipettes, pipette tips, Erlenmeyer flasks, beakers, and graduated cylinders. The specific vessels described herein with respect to an actual reduction to practice of a non-limiting embodiment are polypropylene 96-well microplates and beakers. However, a skilled artisan would understand that the methods and equipment set-up described herein can be modified and adapted, consistent with the present invention, to accommodate and treat alternative vessels.

The surface of the vessel of the first more detailed embodiment may be made from the substrate material itself, e.g., any of the thermoplastic resins listed above. Alternatively, the surface may be a pH protective coating or layer of PECVD deposited SiOₓC_{y}H_{z} or SiNₓC_{y}H_{z}, in which x is from about 0.5 to about 2.4 as measured by X-ray photoelectron spectroscopy (XPS), y is from about 0.6 to about 3 as measured by XPS, and z is from about 2 to about 9 as measured by Rutherford backscattering spectrometry (RBS). Another example is the surface is a barrier coating or layer of PECVD deposited SiOₓ, in which x is from about 1.5 to about 2.9 as measured by XPS, alternatively an oxide or nitride of an organometallic precursor that is a compound of a metal element from Group III and/or Group IV of the Periodic Table, e.g. in Group III: Boron, Aluminum, Gallium, Iridium, Thallium, Scandium, Yttrium, or Lanthanum, (Aluminum and Boron being preferred), and in Group IV: Silicon, Germanium, Tin, Lead, Titanium, Zirconium, Hafnium, or Thorium (Silicon and Tin being preferred). Methods and equipment for depositing these coatings or layers are described in detail in WO2013/071138, published May 16, 2013, which is incorporated herein by reference in its entirety.

Methods according to the first more detailed embodiment employ the use of remote conversion plasma treatment. Unlike direct plasma processing, in the case of remote conversion plasma, neither ions nor electrons of plasma contact the article surface. Neutral species, typically having lower energy, are present in the plasma afterglow, which are sufficiently energetic to react with the article surface, without sputtering or other higher energy chemical reactions induced by ions and electrons. The result of remote conversion plasma is a gentle surface modification without the high energy effects of “direct” plasmas.

Methods according to the first more detailed embodiment employ non-polymerizing gases, such as O₂, N₂, air, O₃, N₂O, H₂, H₂O₂, NH₃, Ar, He, Ne, other non-polymerizing gases, and combinations of any of two or more of the foregoing. These may also include non-polymerizing alcohols, non-polymerizing organic acids and non-polymerizing polar organic solvents. Applicant has successfully carried out experiments wherein the first treatment step (non-polymerizing compound step) used Ar, N₂, Ar/O₂ mix, or N₂/O₂ mix and a pre-treatment conditioning step with Ar. These and other non-polymerizing gases do not necessarily deposit a coating. Rather, they react with the surface to modify the surface, e.g., to form a converted surface, wherein the converted surface has a biomolecule recovery percentage greater than the biomolecule recovery percentage of the untreated surface. For example, the surface reactions may result in new chemical functional groups on the surface, including, but not limited to carbonyl, carboxyl, hydroxyl, nitrile, amide, amine. It is contemplated that these polar chemical groups increase the surface energy and hydrophilicity of otherwise hydrophobic polymers that an untreated surface may typically comprise. While hydrophobic surfaces are generally good binding surfaces for biomolecules, hydrophilic surfaces, which attract water molecules, facilitate the blocking of biomolecules binding to that surface. While not being bound to this theory of operation, it is contemplated that this mechanism prevents biomolecule binding to surfaces.

Optionally, methods according to the first more detailed embodiment may be used to reduce the propensity of a substrate surface to cause biomolecules to adhere thereto. Preferably, the methods will reduce biomolecule adhesion across a wide spectrum of biomolecules, including but not limited to one or more of the following aqueous proteins: mammal serum albumin, for example Bovine Serum Albumin (BSA); Fibrinogen (FBG); Transferrin (TFN), for example blood serotransferrin (or siderophilin, also known as transferrin); lactotransferrin (lactoferrin); milk transferrin; egg white ovotransferrin (conalbumin); and membrane-associated melanotransferrin; Protein A (PrA); Protein G (PrG); Protein A/G; Protein L; Insulin, for example hexameric insulin, monomeric insulin, porcine insulin, human insulin, recombinant insulin and pharmaceutical grades of insulin; Pharmaceutical protein; blood or blood component proteins; or any recombinant form, modification, full length precursor, signal peptide, pro-peptide, or mature variant of these proteins; and a combination of two or more of these.

FIG. 1 is a schematic generic view of remote conversion plasma treatment apparatus 9 of the first more detailed embodiment having common features with each more particular embodiment of FIGS. 2, 6, 7, and 8 for carrying out remote conversion plasma treatment according to the invention. Plasma gas from a fluid source 12 capable of supporting the generation of plasma in the plasma zone 15 having a boundary 20 (plasma is defined here as a visible glow discharge) is introduced via a fluid inlet 13 to a plasma zone 15, and plasma energy from a plasma energy source 18 is provided to the plasma zone 15 to generate plasma having a boundary 20 in the plasma zone 15.

The plasma energy of the first more detailed embodiment broadly can be any type of energy that will ignite plasma in the plasma zone 15. For example, it can be direct current (DC) or alternating current (electromagnetic energy) having a frequency from 3 Hz to 300 GHz. Electromagnetic energy in this range generally includes radio frequency (RF) energy and microwave energy, more particularly characterized as extremely low frequency (ELF) of 3 to 30 Hz, super low frequency (SLF) of 30 to 300 Hz, voice or ultra-low frequency (VF or ULF) of 300 Hz to 3 kHz, very low frequency (VLF) of 3 to 30 kHz, low frequency (LF) of 30 to 300 kHz, medium frequency (MF) of 300 kHz to 3 MHz, high frequency (HF) of 3 to 30 MHz, very high frequency (VHF) of 30 to 300 MHz, ultra-high frequency (UHF) of 300 MHz to 3 GHz, super high frequency (SHF) of 3 to 30 GHz, extremely high frequency (EHF) of 30 to 300 GHz, or any combination of two or more of these frequencies. For example, high frequency energy, commonly 13.56 MHz, is useful RF energy, and ultra-high frequency energy, commonly 2.54 GHz, is useful microwave energy, as two non-limiting examples of commonly used frequencies.

The nature of the optimal applicator 23 of the first more detailed embodiment is determined by the frequency and power level of the energy, as is well known. If the plasma is excited by radio waves, for example, the applicator 23 can be an electrode, while if the plasma is excited by microwave energy, for example, the applicator 23 can be a waveguide.

An afterglow region 24 of the first more detailed embodiment is located outside but near the plasma boundary 20, and contains treatment gas 17. The afterglow region 24 can be the entire treatment volume 10 outside the plasma boundary 20 and within the reaction chamber wall 1 and lid 19, or the afterglow region 24 can be a subset of the treatment volume 10, depending on the dimensions of and conditions maintained in the treatment volume. The treatment gas 17 in the afterglow region 24 is not ionized sufficiently to form plasma, but it is sufficiently energetic to be capable of modifying a surface that it contacts, more so than the same gas composition at the same temperature and pressure in the absence of the plasma.

It will be understood by a skilled person that some gas compositions are sufficiently chemically reactive that they will modify a substrate in the apparatus 9 of the first more detailed embodiment when plasma is absent. The test for whether a region of, or adjacent to, remote conversion plasma treatment apparatus is within the afterglow, for given equipment, plasma, gas feed, and pressure or vacuum conditions producing a visible glow discharge outside the region, is whether a substrate located in the region under the given equipment, plasma, gas feed, and pressure is modified compared to a substrate exposed to the same equipment, gas feed and pressure or vacuum conditions, when no plasma is present in the plasma zone as the result of the absence of or insufficiency of the plasma energy 18 of the first more detailed embodiment.

Remote conversion plasma treatment of the first more detailed embodiment is carried out by providing plasma in the plasma zone 15, which generates an afterglow in the afterglow region or remote conversion plasma (two terms for the same region) 24, which contacts and modifies a substrate placed at least partially in the afterglow region 24.

As one option of the first more detailed embodiment in the remote conversion plasma treatment apparatus, the plasma gas enters the plasma zone, is excited to form plasma, then continues downstream to the afterglow region 24 where it has less energy, is then defined as treatment gas 17, and contacts the substrate. In other words, at least a portion of the gas flows through the plasma zone 15, is energized to form plasma, and continues to the afterglow region 24, becoming more energetic in the plasma zone 15 and less energetic by the time it enters the afterglow region 24 (but still energized compared to the gas before entering the plasma zone 15). Where this option is adopted, the plasma and the afterglow region 24 are in gas communication and at least some of the same gas is fed through both zones. Optionally, as where plasma is not generated in the entire cross-section of flowing gas, some of the gas may bypass the plasma by staying outside the boundary 20 of the plasma zone 15 and still flow through the afterglow region 24, while other gas flows through both the plasma zone 15 and the afterglow region 24.

As another option in the remote conversion plasma treatment apparatus of the first more detailed embodiment, the plasma gas can be different molecules from the treatment gas 17 (though the plasma gas and treatment gas may either have identical compositions or different compositions), and the plasma gas remains in or is fed through only the plasma zone 15 and not the afterglow region 24, while the treatment gas is energized by the plasma gas but is separate from the plasma gas and while in the afterglow region 24 is not energized sufficiently to form plasma.

The nature of the applicator 23 of the first more detailed embodiment can vary depending on the application conditions, for example the power level and frequency of the plasma energy 18. For example, the applicator can be configured as an electrode, antenna, or waveguide.

Optionally, a shield 16 may be placed between the plasma and at least a portion of the substrate 14 in the treatment area of the first more detailed embodiment to prevent the plasma from contacting or coming undesirably close to the substrate 14 or unevenly affecting the substrate 14. For one example, the optional shield 16 in FIG. 1 can be perforated to allow gas flow through it, particularly flow of the neutral species forming the afterglow, but the shield 16 is configured or equipped, suitably for the choice of plasma-forming energy, to prevent the plasma from penetrating the shield of the first more detailed embodiment. For example, the perforations may be sized or the shield can be electrically biased such that the plasma-forming energy or the plasma cannot pass through it. This arrangement has the advantage that, if the plasma zone has a substantial area intersecting with the shield, the substantial area optionally is flattened so the plasma boundary 20 has a “flat spot” 26, illustrated in FIG. 1 of the first more detailed embodiment, which can be placed parallel to the surface of the substrate to be treated so they are equidistant over a substantial area, instead of the plasma terminating in a tapered tail that extends much closer to one portion of the substrate 14 than to other parts of the substrate 14 not aligned with the tail, illustrated in FIG. 8 of the first more detailed embodiment.

Another shield option of the first more detailed embodiment is that the shield can be made such that it passes neither gas nor plasma, serving as an obstruction of the direct path between some or all of the plasma and some or all of the treatment area. The obstruction can fill less than all of the gas cross-section flowing from the plasma zone 15 to the afterglow region 24, so non-ionized gas can flow around the shield and reach the afterglow region 24 by a circuitous path, while plasma cannot either circumvent or pass through it.

Yet another shield option of the first more detailed embodiment is that the substrate 14 to be treated can be positioned in the apparatus during treatment such that one portion of a substrate 14 that can withstand contact with plasma is exposed to the plasma, shielding from the plasma another portion of the substrate 14 or another substrate receiving remote conversion plasma treatment.

Still another shield option of the first more detailed embodiment is that the gas flow path through the plasma and treatment area can be sharply bent, for example turning a 90 degree corner between the plasma and treatment area, so the wall of the apparatus itself shields the treatment area from line-of-sight relation to the plasma under certain treatment conditions.

The substrate orientation in the treatment volume of the first more detailed embodiment can vary, and the substrate, applicator, gas and vacuum sources can optionally be arranged to provide either substantially even or uneven exposure to remote conversion plasma across a substrate.

Another option in the first more detailed embodiment is that the substrate itself can serve as the reactor wall or a portion of the reactor wall, so treatment gas 17 introduced into reactor treats the portion of the substrate serving as the reactor wall.

Another option in the first more detailed embodiment is the introduction of a second non-polymerizing gas, functioning as diluent gas, into the reactor, in addition to the non-polymerizing compound or water vapor which is the active agent of the treatment gas 17. Diluent gases are defined as gases introduced at the fluid inlet 13 that do not materially interact with the substrate 14 to the extent they find their way into the treatment gas 17, given the treatment apparatus and conditions applied. Diluent gases can either participate or not participate in formation of the plasma. The diluent gas can be introduced through the inlet 13 or elsewhere in the reactor. Diluent gases can be added at a rate from 1% to 10,000% by volume, optionally 10% to 1000% by volume, optionally 100% to 1000% by volume, of the rate of addition of the non-polymerizing compound or water vapor.

As another option in the first more detailed embodiment, some or all of the non-polymerizing compound or water vapor can be added to the treatment volume 10 in such a manner as to bypass the plasma zone 15 en route to the treatment gas 17.

FIG. 2 of the first more detailed embodiment shows another embodiment of the apparatus of FIG. 1. The apparatus again can be used for carrying out the remote conversion plasma treatment according to the first more detailed embodiment. The chamber of this embodiment comprises a treatment volume 10 defined and enclosed by a reaction chamber wall 11, which optionally is not electrically conductive. The treatment volume 10 is supplied with a fluid source 12 (in this instance, a tubular fluid inlet 13 projecting axially into the treatment volume 10, however other fluid sources are contemplated, e.g., “shower head” type fluid sources). Optionally, the treatment volume 10 can be defined by a treatment chamber wall 11 or by the lumen within a vessel or other article to be treated. Feed gases are fed into the treatment volume 10. The plasma reaction chamber comprises as an optional feature a vacuum source 22 for at least partially evacuating the treatment volume 10 compared to ambient pressure, for use when plasma treating at reduced pressure, although plasma treating under suitable conditions at ambient atmospheric pressure or at a pressure higher than ambient atmospheric pressure is also contemplated.

The plasma reaction chamber also comprises an optional outer applicator 23, here in the form of an electrode surrounding at least a portion of the plasma reaction chamber. A radio frequency (RF) plasma energy source 18 is coupled to the reaction chamber by an applicator 23 and provides power that excites the gases to form plasma. The plasma forms a visible glow discharge 20 that optionally is limited to a close proximity to the fluid source 12.

Microplates 14 optionally can be oriented such that the surfaces of the microplates 14 on which treatment is desired (the surface that is configured and intended to contact/hold a biomolecule-containing solution) faced the fluid source 12. However, the surfaces to be treated can also or instead face away from the fluid source 12, as shown in FIG. 2. In addition, in the illustrated embodiment the microplate 14 is shielded with a shield 16 to block the microplate 14 from being in the direct “line of sight” of (i.e. having an unobstructed path to) the fluid source 12. As a non-limiting example, the respective surfaces of the microplates 14 can be positioned a horizontal distance of approximately 2.75 inches (7 cm) from the fluid source, although operation is contemplated with placement of the microplate 14 surfaces at a horizontal distance of from ½ to 10 inches (1 to 25 cm), optionally 2.5 to 5.5 inches (6 to 14 cm) from the fluid source. In this manner, the process relies on remote conversion plasma (as opposed to direct plasma) to treat the microplates' 14 surfaces. In this non-limiting example, the system has a capacity of 20 parts (microplates) per batch at a total process time of eight minutes per batch.

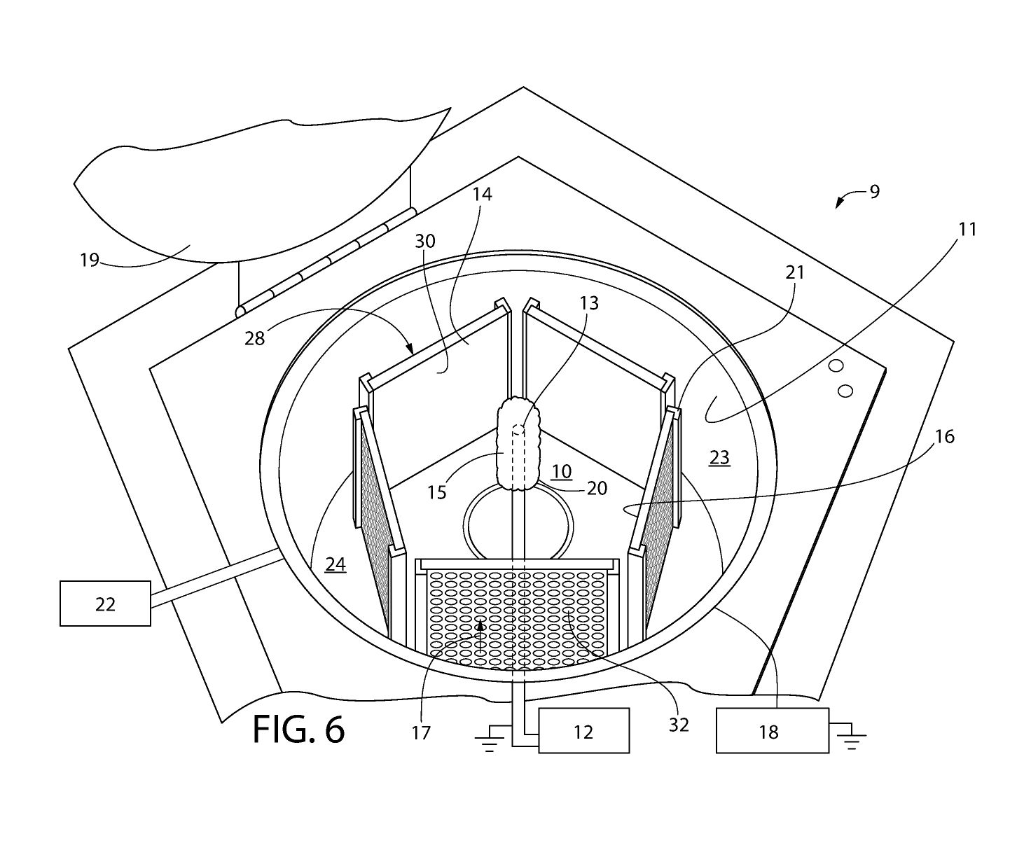

FIG. 6 of the first more detailed embodiment shows another embodiment of the apparatus of FIG. 1. The process used to treat the microplates in FIG. 6 uses a radio-frequency (RF) plasma system. The system has a gas delivery input, a vacuum pump and RF power supply with matching network. The microplates are shown oriented with the front surfaces containing the wells 32 facing away from and shielded from the plasma along the perimeter of the chamber.

These details are illustrated in FIG. 6 of the first more detailed embodiment, where there is shown another exemplary setup having all the elements of the apparatus of FIG. 2 of the first more detailed embodiment for use in a plasma reaction chamber for carrying out remote conversion plasma treatment according to the first more detailed embodiment. The chamber of the first more detailed embodiment comprised a treatment volume 10 defined and enclosed by a reaction chamber wall 11 having a fluid source 12 (in this instance, a tubular fluid inlet 13 projecting axially into the treatment volume 10, however other fluid sources are contemplated, e.g., “shower head” type fluid sources). The reaction chamber wall 11 in this embodiment was provided with a removable lid 13 that is openable to allow substrates to be inserted or removed and sealable to contain the process and, optionally, evacuate the treatment volume. In the first more detailed embodiment, the fluid source 12 was made of metallic material, electrically grounded, and also functioned as an applicator, in the form of an inner electrode. As is well known, the plasma of the first more detailed embodiment alternatively can be generated without an inner electrode.

Feed gases were fed into the treatment volume 10. The plasma reaction chamber comprised an optional feature of a vacuum source 22 for at least partially evacuating the treatment volume 10. The plasma reaction chamber wall 11 also functioned as an applicator 23 in the form of an outer applicator or electrode surrounding at least a portion of the plasma reaction chamber. A plasma energy source 18, in this instance a radio frequency (RF) source, was coupled to applicators 23 defined by the reaction chamber wall 24 and the fluid source 12 to provide power that excited the gases to form plasma. The plasma zone 15 formed a visible glow discharge that was limited by the plasma boundary 20 in close proximity to the fluid source 12. The afterglow region also known as a remote conversion plasma region 24 is the region radially or axially outside the boundary 20 of the visible glow discharge and extending beyond the substrates treated.

Microplates 14 having front surfaces 28 and back surfaces 30 were oriented such that the wells 32 on the front surfaces of the microplates 14 on which treatment was desired (the front surface that is configured and intended to contact/hold a biomolecule-containing solution) faced away from the fluid source 12 and the back surfaces 30 faced toward the fluid source 12. The front surfaces 28 of the microplates 14 were shielded by their own back surfaces 30 to block the microplate front surfaces 28 from being in the direct “line of sight” of the fluid source 12. In this manner, the process relied on remote conversion plasma (as opposed to direct plasma) to treat the surfaces of the wells 32.

FIG. 7 of the first more detailed embodiment shows another embodiment of the apparatus of FIG. 1, having corresponding features. The embodiment of FIG. 7 provides a “shower head” fluid inlet 13 and a plate electrode as the applicator 23 that provide more uniform generation and application of treatment gas 17 over a wider area of the substrate 14.

FIG. 8 of the first more detailed embodiment shows another embodiment of the apparatus of FIG. 1, having corresponding features. The embodiment of FIG. 8 provides microwave plasma energy 18 delivered through an applicator 23 configured as a waveguide. In this embodiment the plasma zone 15 and substrate support 21 are provided in separate vessels connected by a conduit.

Optionally in the first more detailed embodiment, the converted surface has a biomolecule recovery percentage greater than the biomolecule recovery percentage of the untreated surface for at least one of Bovine Serum Albumin (BSA); Fibrinogen (FBG); Transferrin (TFN), for example blood serotransferrin (or siderophilin, also known as transferrin); lactotransferrin (lactoferrin); milk transferrin; egg white ovotransferrin (conalbumin); and membrane-associated melanotransferrin; Protein A (PrA); Protein G (PrG); Protein A/G; Protein L; Insulin, for example hexameric insulin, monomeric insulin, porcine insulin, human insulin, recombinant insulin and pharmaceutical grades of insulin; pharmaceutical protein; blood or blood component proteins; or any recombinant form, modification, full length precursor, signal peptide, propeptide, mature variant of these proteins and a combination of two or more of these.

In one optional embodiment of the first more detailed embodiment, a plasma treatment process comprises, consists essentially of, or consists of the following two steps using remote conversion plasma: (1) an oxygen plasma step (or more generically, a non-polymerizing compound plasma step) followed by (2) a water vapor plasma step. It should be understood that additional steps prior to, between or after the aforementioned steps may be added and remain within the scope of the first more detailed embodiment. Further, it should also be understood that the oxygen plasma step may utilize alternative gases to oxygen, including but not limited to nitrogen or any non-polymerizing gases listed in this specification.

Optional process parameter ranges for the first treatment step (non-polymerizing compound plasma step) and second treatment step (water vapor plasma step) of the first more detailed embodiment are set forth in Table 1 of the first more detailed embodiment.

TABLE 1
Process parameter ranges for plasma treatment

| Non-Polymerizing | | Water Vapor Plasma | |
|---|---|---|---|
| Compound Plasma Step | | Step | |
| Power (W) | 50-600 | Power (W) | 50-600 |
| Gas Flow rate (sccm) | 5-50 | H₂O Flow rate (sccm) | 1-10 |
| Time (minutes) | 0.5-5 | Time (minutes) | 0.05-5 |
| Pressure (mTorr) | 0-1,000 | Pressure (mTorr) | 0-5,000 |

Optionally, no pretreatment step is required prior to the non-polymerizing gas plasma step.

Optionally, in the first more detailed embodiment, the remote conversion plasma used to treat a substrate surface may be RF generated plasma. Optionally, plasma enhanced chemical vapor deposition (PECVD) or other plasma processes may be used consistent with the first more detailed embodiment.

Optionally, the treatment volume in a plasma reaction chamber may be from 100 mL to 50 liters, preferably 8 liters to 20 liters for certain applications. Optionally, the treatment volume may be generally cylindrical, although other shapes and configurations are also contemplated.

Various aspects will be illustrated in more detail with reference to the following Examples, but it should be understood that the first more detailed embodiment is not deemed to be limited thereto.

### Testing of all Embodiments

The following protocol was used to test the plates in all embodiments, except as otherwise indicated in the examples:

Purpose: The purpose of this experiment was to determine the amount of protein binding over time to a surface coated microtiter plate.

Materials: BIOTEK® Synergy H1 Microplate Reader and BIOTEK Gen5® Software, MILLIPORE® MILLI-Q® Water System (sold by Merck KGAA, Darmstadt, Germany), MILLIPORE® Direct Detect Spectrometer, ALEXA FLUOR® 488 Labeled Proteins (Bovine Serum Albumin (BSA), Fibrinogen (FBG), Transferrin (TFN), Protein A (PrA) and Protein G (PrG), sold by Molecular Probes, Inc., Eugene, Oreg. USA), 10× Phosphate Buffered Saline (PBS), NUNC® Black 96-well Optical Bottom Plates, 1 L Plastic Bottle, 25-100 mL Glass Beakers, Aluminum Foil, 1-10 mL Pipette, 100-1000 μL Pipette, 0.1-5 μL Pipette, 50-300 μL Multichannel Pipette.

The selected proteins, one or more of those listed above, were tested on a single surface coated microplate. Each protein was received as a fluorescently labeled powder, labeled with ALEXA FLUOR® 488:
5 mg of BSA: 66,000 Da
5 mg of FBG: 340,000 Da
5 mg of TFN: 80,000 Da
1 mg of PrA: 45,000 Da
1 mg of PrG: 20,000 Da

Once received, each vial of protein was wrapped in aluminum foil for extra protection from light and labeled accordingly, then placed into the freezer for storage.

A solution of 1×PBS (phosphate buffer solution) was made from a stock solution of 10×PBS: 100 mL of 10×PBS was added to a plastic 1 L bottle, followed by 900 mL of distilled water from the MILLIPORE® Q-pod, forming 1X PBS. Using a 100-1000 μL pipette, 1000 μL of 1×PBS was pipetted into each vial of protein separately, to create protein solutions. Each vial was then inverted and vortexed to thoroughly mix the solution.

Each protein was then tested on the MILLIPORE® Direct Detect to get an accurate protein concentration. Using a 0.1-5 μL pipette, a 2 μL sample of PBS was placed on the first spot of the Direct Detect reading card and marked as a blank in the software. A 2 μL sample of the first protein was then placed onto the remaining 3 spots and marked as samples. After the card was read, an average of the 3 protein concentrations was recorded in mg/mL. This is repeated for the remaining 4 proteins. The protein solutions are then placed into the refrigerator for storage.

A standard curve was prepared with 1×PBS for each protein. The standard curve started at 25 nM and a serial 2× dilution was performed to obtain the other tested concentrations, for example one or more of 12.5 nM, 6.25 nM, 3.125 nM and 1.5625 nM. The 12.5 nM solution prepared from the standard curve was used for testing.

Once the dilutions for all tested proteins were done, the standard curve for each protein was prepared and tested as follows. 25 100-mL glass beakers were set into rows of 5. Each beaker was wrapped in aluminum foil and labeled with the name of the protein the curve corresponded to and the concentration of the solution in the beaker. Row 1 was the standard curve for BSA; row 2, FBG; row 3, TFN; row 4, PrA; row 5, PrG. Therefore the first row was labeled as follows: BSA 25 nM, BSA 12.5 nM, BSA 6.25 nM, BSA 3.125 nM, BSA 1.56 nM.

After a standard curve was made, it was tested using the microplate reader, then the next standard curve was made and tested, and so on.

The BIOTEK® Synergy H1 microplate reader and BIOTEK Gen5® software were used for analysis.

After the first standard curve was prepared, it was ready to be tested on the Synergy H1. Using a 50-300 μL multichannel pipette, 200 μL of 1×PBS was pipetted into wells A1-A4 of a black optical bottom microplate. Then, 200 μL of the 25 nM solution was pipetted into wells B1-B4, 200 μL of 12.5 nM solution was pipetted into wells C1-C4, 200 μL of 12.5 nM solution was pipetted into wells D1-D4, 200 μL of 12.5 nM solution was pipetted into wells E1-E4, 200 μL of 12.5 nM solution was pipetted into wells F1-F4, and 200 μL of 12.5 nM solution was pipetted into wells G1-G4. A similar procedure was used to fill the wells with other dilutions of the protein solution.

Once the microplate was filled with solution, it was wrapped in aluminum foil and the sections and time points were labeled.

After 1.5 hours, using a 50-300 μL multichannel pipette and poking through the aluminum foil, 200 μL of BSA solution was pipetted from the wells in the 1.5 hr column (column 1) and placed into a black optical bottom microplate. The black microplate was placed into the microplate tray. The other four proteins were then read the same way by opening their corresponding experiments. The same thing was done after 2.5 hours, 4.5 hours and 24 hours. After the 24 hr read, “Plate→Export” was then selected from the menu bar. An excel spreadsheet will appear and can then be saved in the desired location with the desired name.

Using the data produced by the BIOTEK Gen5® software, the 12.5 nM solution concentrations from both the standard curve and SPL1 were averaged. The concentrations in the 4 wells at 1.5 hr were averaged. This was then done for 2.5 hr, 4.5 hr and 24 hr also. The average concentration at each time point was then divided by the average concentration of The 12.5 nM solution from the beginning and multiplied by 100 to get a percent recovery at each time point:

% Recovery@1.5 hr=[AVG. BSA 1.5 hr]/[AVG 12.5 nM solution]*100

### EXAMPLES

### Example 1 of the First More Detailed Embodiment

Polypropylene 96-well microplates were plasma treated according to an optional aspect of the first more detailed embodiment. The process used to treat the microplates used a radio-frequency (RF) plasma system. The system had a gas delivery input, a vacuum pump and RF power supply with matching network. The microplates were oriented facing away from and shielded from the plasma along the perimeter of the chamber. These details are illustrated in FIG. 2. The shielding resulted in remote plasma treatment in which the ratio between the radiant density at the remote points on the surfaces of the microplates and the brightest point of the plasma discharge was less than 0.25.

The two step remote conversion plasma process used according to this non-limiting example is summarized in Table 2 of the first more detailed embodiment:

TABLE 2
Process parameters for plasma treatment per Example
1 of the first more detailed embodiment

| Process Step 1- | | Process Step 2- | |
|---|---|---|---|
| Conditioning Plasma | | Conversion Plasma | |
| Power (W) | 500 | Power (W) | 500 |
| O₂ Flow rate (sccm) | 50 | H₂O Flow rate (sccm) | 5 |
| Time (minutes) | 5 | Time (minutes) | 3 |
| Pressure (mTorr) | 50 | Pressure (mTorr) | 80-120 |

The biomolecule binding resistance resulting from this remote conversion plasma process of the first more detailed embodiment on the surface of the treated microplates was analyzed by carrying out the Testing of All Embodiments. The percent recovery is the percentage of the original concentration of the protein remaining in solution, i.e., which did not bind to the surface of a microplate.

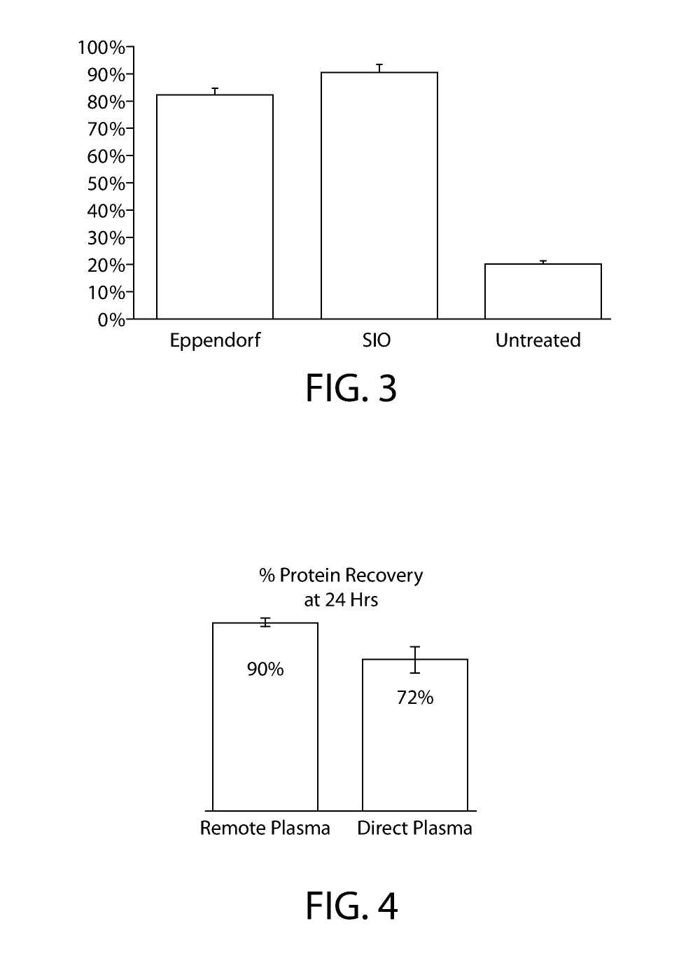

In this testing, samples of three different types of microplates were tested for percent recovery. The samples included: (1) untreated polypropylene microplates (“Untreated” samples); (2) polypropylene microplates treated according to the first more detailed embodiment described in Example 1 of this specification (“SIO” samples); and (3) EPPENDORF brand microplates (“EPPENDORF” samples). The bar chart in FIG. 3 shows the results of this comparative testing. As FIG. 3 of the first more detailed embodiment illustrates, the SIO samples had a 60% increase in biomolecule recovery compared to the Untreated Samples and an 8-10% increase in biomolecule recovery compared to the EPPENDORF samples.

Accordingly, remote conversion plasma treatment according to the first more detailed embodiment has been demonstrated to result in lower biomolecule adhesion (or the inverse, higher biomolecule recovery) than other known methods. In fact, the comparative data of the SIO samples and the EPPENDORF samples were particularly surprising, since EPPENDORF has been considered the industry standard in protein resistant labware. The SIO samples' 8-10% increase in efficacy compared to the EPPENDORF samples represents a marked improvement compared to the state of the art.

### Example 2 of the First More Detailed Embodiment

In this example of the first more detailed embodiment, the SIO samples of Example 1 were compared to the same microplates that were treated with same process steps and conditions as the SIO, except (and this is an important exception), the second samples were treated with direct plasma instead of remote conversion plasma (the “Direct Plasma” samples). Surprisingly, as shown in FIG. 4, the Direct Plasma samples had a biomolecule recovery percentage after 24 hours of 72%, while the SIO samples (which were treated under the same conditions/process steps except by remote conversion plasma) had a biomolecule recovery percentage after 24 hours of 90%. This remarkable step change demonstrates the unexpected improvement resulting solely from use of remote conversion plasma of the first more detailed embodiment in place of direct plasma.

### Example 3 of the First More Detailed Embodiment

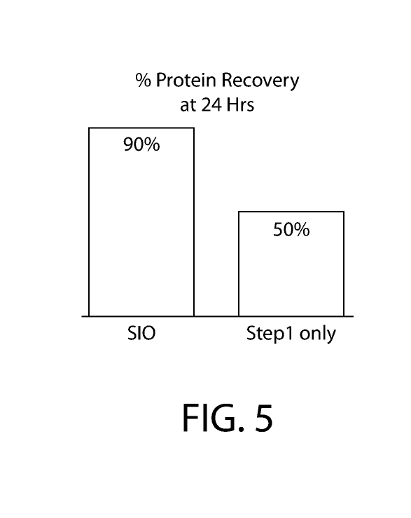

In this example of the first more detailed embodiment, the SIO samples of Example 1 were compared to the same microplates that were treated with only the first treatment step of the method of the first more detailed embodiment (i.e., the non-polymerizing compound plasma step or conditioning plasma treatment) without the second treatment step (water vapor plasma step or conversion plasma treatment) (“Step 1 Only” samples). As shown in FIG. 5, Step 1 Only samples had a biomolecule recovery percentage after 24 hours at about 25° C. (the aging of all protein samples in this specification is at 25° C. unless otherwise indicated) of 50%, while the SIO samples (which were treated under the same conditions/process steps except also by remote conversion plasma) had a biomolecule recovery percentage after 24 hours of 90%. Accordingly, using both steps of the method according to embodiments of the first more detailed embodiment results in significantly improved biomolecule recovery percentage than using only the first treatment step alone.

### Example 4 (Prophetic) of the First More Detailed Embodiment

A further contemplated optional advantage of the first more detailed embodiment is that it provides high levels of resistance to biomolecule adhesion without a countervailing high extractables profile. For example, a leading brand of labware made by EPPENDORF is resistant to biomolecule adhesion by virtue of a chemical additive, which has a propensity to extract from the substrate and into a solution in contact with the substrate. By contrast, the first more detailed embodiment does not rely on chemical additives mixed into a polymer substrate to give the substrate its biomolecule adhesion resistant properties. Moreover, processes according to the first more detailed embodiment do not result in or otherwise cause compounds or particles to extract from a treated substrate. Applicant has further determined that the pH protective chemistries described herein, when deposited on a substrate (providing a surface for treatment), do not result in or otherwise cause compounds or particles to extract from a treated surface.

Accordingly, in one optional aspect, the present invention (in the first more detailed embodiment described herein) is directed to a method for treating a surface of a substrate, also referred to as a material or workpiece, to form a converted surface wherein the converted surface has a biomolecule recovery percentage greater than the biomolecule recovery percentage of the surface prior to treatment according to the method, and wherein the method does not materially increase the extractables profile of the substrate. Applicants contemplate that this would bear out in actual comparative tests.

### Example 5 of the First More Detailed Embodiment

A test similar to Example 1 of the first more detailed embodiment was carried out to compare the biomolecule recovery from untreated polypropylene (UTPP) laboratory beakers, remote conversion plasma treated polypropylene (TPP) laboratory beakers according to the first more detailed embodiment, and untreated glass laboratory beakers. The biomolecules used were 12 nM dispersions of lyophilized BSA, FBG, TFN, PrA, and PrG.

In a first trial of the first more detailed embodiment, the biomolecule dispersion was made up in the beaker and aspirated several times to mix it. The biomolecule recovery was measured in relative fluorescence units (RFU). The initial RFU reading (0 min) was taken to establish a 100% recovery baseline, then the biomolecule dispersion in the beaker was stirred for 1 min with a pipet tip, after which it was allowed to remain on the laboratory bench undisturbed for the remainder of the test. The biomolecule recovery was measured initially, then a sample was drawn and measured for percentage biomolecule recovery at each 5-minute interval. The results are shown in Table 3.

TABLE 3
Polypropylene beaker trial of the first more detailed embodiment

| UTPP BSA | | UTPP FBG | | UTPP TFN | | UTPP PrA | | UTPP PrG | |
|---|---|---|---|---|---|---|---|---|---|
| Time (min) | % R | Time (min) | % R | Time (min) | % R | Time (min) | % R | Time (min) | % R |
| 0 | 100% | 0 | 100% | 0 | 100% | 0 | 100% | 0 | 100% |
| 1 | 99% | 1 | 99% | 1 | 98% | 1 | 100% | 1 | 101% |
| 5 | 98% | 5 | 97% | 5 | 94% | 5 | 98% | 5 | 99% |
| 10 | 99% | 10 | 99% | 10 | 93% | 10 | 99% | 10 | 100% |
| 15 | 98% | 15 | 92% | 15 | 90% | 15 | 97% | 15 | 99% |
| 20 | 99% | 20 | 98% | 20 | 88% | 20 | 98% | 20 | 101% |
| 25 | 99% | 25 | 97% | 25 | 85% | 25 | 96% | 25 | 98% |
| 30 | 98% | 30 | 96% | 30 | 85% | 30 | 96% | 30 | 99% |

| TPP BSA | | TPP FBG | | TPP TFN | | TPP PrA | | TPP PrG | |
|---|---|---|---|---|---|---|---|---|---|
| Time (min) | % R | Time (min) | % R | Time (min) | % R | Time (min) | % R | Time (min) | % R |
| 0 | 100% | 0 | 100% | 0 | 100% | 0 | 100% | 0 | 100% |
| 1 | 104% | 1 | 101% | 1 | 100% | 1 | 104% | 1 | 104% |
| 5 | 104% | 5 | 100% | 5 | 96% | 5 | 104% | 5 | 104% |
| 10 | 106% | 10 | 101% | 10 | 96% | 10 | 104% | 10 | 104% |
| 15 | 104% | 15 | 99% | 15 | 94% | 15 | 102% | 15 | 104% |
| 20 | 106% | 20 | 100% | 20 | 94% | 20 | 104% | 20 | 104% |
| 25 | 103% | 25 | 98% | 25 | 91% | 25 | 102% | 25 | 101% |
| 30 | 105% | 30 | 98% | 30 | 90% | 30 | 103% | 30 | 103% |

A second trial of the first more detailed embodiment, with results shown in Table 4, was carried out in the same manner as the first trial except that glass beakers, not treated according to the first more detailed embodiment, were used as the substrate.

TABLE 4
Glass beaker trial of the first more detailed embodiment

| Glass BSA | | Glass FBG | | Glass TFN | | Glass PrA | | Glass PrG | |
|---|---|---|---|---|---|---|---|---|---|
| Time (min) | % R | Time (min) | % R | Time (min) | % R | Time (min) | % R | Time (min) | % R |
| 0 | 100% | 0 | 100% | 0 | 100% | 0 | 100% | 0 | 100% |
| 1 | 100% | 1 | 100% | 1 | 100% | 1 | 105% | 1 | 99% |
| 4 | 99% | 4 | 88% | 4 | 98% | 4 | 103% | 4 | 97% |
| 7 | 100% | 7 | 99% | 7 | 97% | 7 | 104% | 7 | 98% |
| 8 | 98% | 8 | 98% | 8 | 96% | 8 | 102% | 8 | 96% |
| 10 | 98% | 10 | 98% | 10 | 95% | 10 | 100% | 10 | 97% |
| 15 | 96% | 15 | 95% | 15 | 92% | 15 | 100% | 15 | 94% |
| 20 | 97% | 20 | 96% | 20 | 92% | 20 | 101% | 20 | 93% |
| 25 | 96% | 25 | 93% | 25 | 88% | 25 | 95% | 25 | 91% |
| 30 | 94% | 30 | 93% | 30 | 87% | 30 | 98% | 30 | 92% |

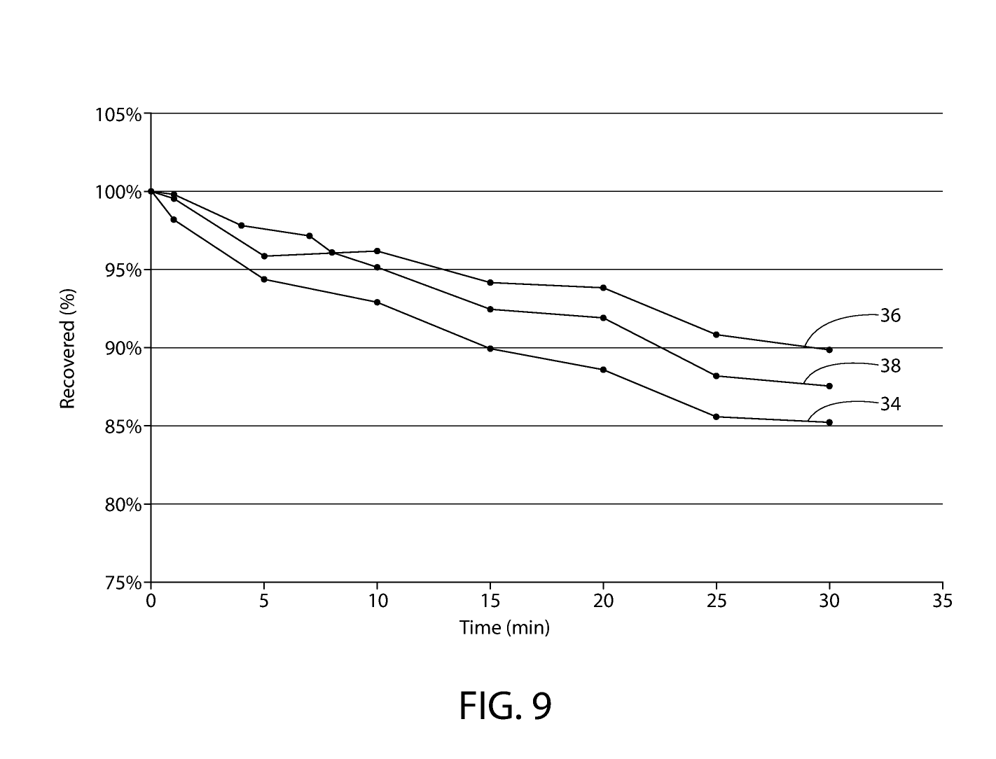

FIG. 9 of the first more detailed embodiment plots the TFN results in Tables above, showing plots 34 for the untreated polypropylene beaker, 36 for the treated polypropylene beaker, and 38 for glass. As FIG. 9 shows, the treated polypropylene beaker provided the highest biomolecule recovery after 10 to 30 minutes, glass produced a lower biomolecule recovery after 10 to 30 minutes, and the untreated polypropylene beaker provided the lowest biomolecule recovery at all times after the initial measurement.

### Example 6 of the First More Detailed Embodiment

A test similar to Example 1 of the first more detailed embodiment was carried out to compare the biomolecule recovery from multiwell polypropylene plates of two types, versus protein concentration, after 24 hours of contact between the protein and the plate. “SIO” plates were molded from polypropylene and plasma treated according to Example 1. “CA” (Competitor A) plates were commercial competitive polypropylene plates provided with a coating to provide reduced non-specific protein binding.

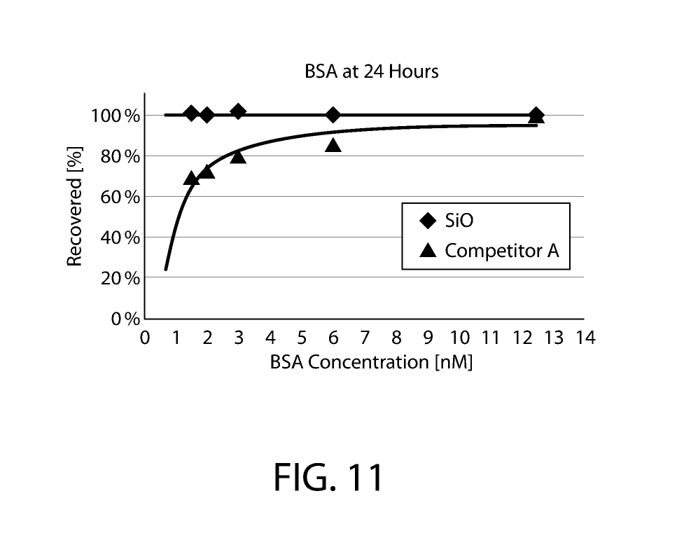

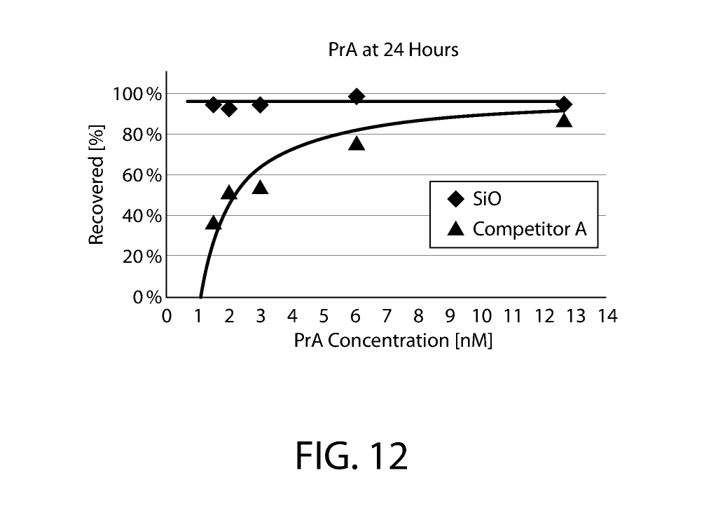

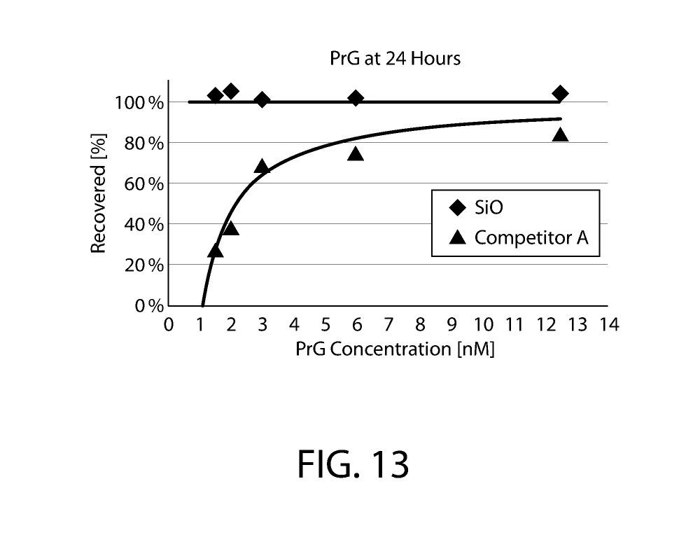

The results are provided in Table 5 and FIGS. 11-13 showing that essentially all the protein of each type was recovered from the SIO plates at all tested concentrations, so the recovery was independent of concentration. In contrast, the protein recovery from the CA plates depended strongly on the concentration, particularly at lower concentrations

TABLE 5
RECOVERY @ 24 hrs in %
(96-Well 1000 μL Deep Well Plate)

| Concentration | | | | |
|---|---|---|---|---|
| (nM) | Plate | BSA | PrA | PrG |
| 1.5 | SIO | 101 | 94 | 103 |
| 2 | SIO | 100 | 92 | 105 |
| 3 | SIO | 102 | 94 | 101 |
| 6 | SIO | 100 | 98 | 102 |
| 12.5 | SIO | 100 | 94 | 104 |
| 1.5 | CA | 70 | 37 | 27 |
| 2 | CA | 73 | 52 | 38 |
| 3 | CA | 80 | 54 | 69 |
| 6 | CA | 86 | 76 | 75 |
| 12.5 | CA | 100 | 87 | 84 |

### Example 7 of the First More Detailed Embodiment

A test similar to Example 1 of the first more detailed embodiment was carried out to compare the biomolecule recovery from “SIO” plates and “CA” plates of the types described in Example 6. The biomolecules used were 1.5 or 3 nM dispersions of lyophilized BSA, FBG, TFN, PrA, and PrG.

The conditions and results are shown in Table 6. For the BSA, PrA, PrG, and TFN proteins, the SIO plates provided substantially superior protein recovery, compared to the CA plates. For the FBG protein, the SIO plates provided better protein recovery than the CA plates.

TABLE 6
RECOVERY @ 72 hrs in %
(96 Well 350 μL (SiO) or 500 μL (CA) Shallow Plate)

| Concentration | | | | | | |
|---|---|---|---|---|---|---|
| (nM) | Plate | BSA | FBG | TFN | PrA | PrG |
| 1.5 | SIO | 104 | 85 | 79 | 99 | 101 |
| 3 | SIO | 100 | 85 | 71 | 93 | 98 |
| 1.5 | CA | 69 | 77 | 45 | 44 | 39 |
| 3 | CA | 74 | 83 | 38 | 60 | 66 |

### Example 8

A test similar to Example 7 of the first more detailed embodiment was carried out to compare 96-well, 500 μL SIO and CA plates. The conditions and results are shown in Table 7. For the BSA, PrA, PrG, and TFN proteins, as well as the 1.5 nM concentration of FBG, the SIO plates provided substantially superior protein recovery, compared to the CA plates. The 3 nM concentration of FBG was anomalous.

TABLE 7
RECOVERY @ 72 hrs in %
(96 Well 500 μL Deep Well Plate)

| Concentration | | | | | | |
|---|---|---|---|---|---|---|
| (nM) | Plate | BSA | FBG | TFN | PrA | PrG |
| 1.5 | SIO | 101 | 85 | 68 | 93 | 104 |
| 3 | SIO | 96 | 74 | 71 | 91 | 104 |
| 1.5 | CA | 69 | 77 | 45 | 44 | 39 |
| 3 | CA | 74 | 83 | 38 | 60 | 66 |

### Example 9

A test similar to Example 7 of the first more detailed embodiment was carried out to compare 96-well, 1000 μL SIO and CA plates. The conditions and results are shown in Table 8. For the BSA, PrA, and PrG proteins, the SIO plates provided substantially superior protein recovery, compared to the CA plates. The FBG proteins did not demonstrate substantially superior protein recovery.

TABLE 8
RECOVERY @ 72 hrs in %
(96 Well 1000 μL Deep Well Plate)

| Concentration | | | | | | |
|---|---|---|---|---|---|---|
| (nM) | Plate | BSA | FBG | TFN | PrA | PrG |
| 1.5 | SIO | 101 | 51 | 64 | 99 | 100 |
| 3 | SIO | 99 | 63 | 62 | 99 | 102 |
| 1.5 | CA | 84 | 76 | 44 | 38 | 44 |
| 3 | CA | 81 | 83 | 46 | 63 | 52 |

### Example 10

A test similar to Example 7 of the first more detailed embodiment was carried out to compare 384 Well 55 μL (SiO) vs 200 μL (CA) shallow plates. The conditions and results are shown in Table 8. For the BSA, PrA, and PrG proteins, the SIO plates provided substantially superior protein recovery, compared to the CA plates. The FBG proteins did not demonstrate substantially superior protein recovery.

TABLE 9
RECOVERY @ 72 hrs in %
(384 Well 55 μL (SiO) or 200 μL (CA) Shallow Plate)

| Concentration | | | | | | |
|---|---|---|---|---|---|---|
| (nM) | Plate | BSA | FBG | TFN | PrA | PrG |
| 1.5 | SIO | 97 | 38 | 92 | 71 | 102 |
| 3 | SIO | 102 | 57 | 87 | 92 | 104 |
| 1.5 | CA | 34 | 58 | 32 | 27 | 14 |
| 3 | CA | 63 | 62 | 39 | 37 | 28 |

### Example 11

A test similar to Example 1 of the first more detailed embodiment was carried out to compare the SIO treated plates of the first more detailed embodiment to polypropylene plates treated with StabilBlot® BSA Blocker, a commercial treatment used to reduce BSA protein adhesion, sold by SurModics, Inc., Eden Prairie, Minn., USA. The conditions and results are shown in Table 10, where SIO is the plate according to Example 1, Plate A is a polypropylene plate treated with 5% BSA blocker for one hour and Plate B is a polypropylene plate treated with 1% BSA blocker for one hour. Except for FBG protein, the present invention again provided superior results compared to the BSA blocker plates.

TABLE 10
RECOVERY @ 24 hrs in %
(3 nM in buffer)

| Concentration | | | | | | |
|---|---|---|---|---|---|---|
| (nM) | Plate | BSA | FBG | TFN | PrA | PrG |
| 3 | SIO | 102 | 69 | 79 | 96 | 104 |
| 3 | A | 97 | 85 | 71 | 93 | 102 |
| 3 | B | 94 | 84 | 66 | 70 | 75 |

A: 5% BSA blocker passivation of 1000 μL, treated 1 hr;
B: 1% BSA blocker passivation of 1000 μL, treated 1 hr;

### Example 12

A test similar to Example 7 of the first more detailed embodiment was carried out to compare the protein recovery rates of SIO treated plates in accordance with Example 1 over longer periods of time—from 1 to 4 months. The conditions and results are shown in Table 12, which illustrates that roughly uniform resistance to protein adhesion was observed for all of the proteins over a substantial period.

TABLE 12
RECOVERY in %

| Time | | | | | |
|---|---|---|---|---|---|
| (month) | BSA | FBG | TEN | PrA | PrG |
| Initial | 97 | 80 | 63 | 98 | 101 |
| 1 | 95 | — | 66 | 81 | 100 |
| 2 | 94 | 85 | 61 | 87 | 96 |
| 3 | 92 | 77 | 79 | 87 | 94 |
| 4 | 97 | 74 | 77 | 85 | 94 |

### Example 13

The uniformity of binding among the different wells of a single plate was tested using two 96-well plates with deep (500 μL) wells, one and SIO plate prepared according to Example 1 except testing 2 nM PrA protein after two hours in all 96 wells, and the other a Competitor A plate, again testing 2 nM PrA protein after two hours in all 96 wells. The protein recovery from each well on one plate was measured, then averaged, ranged (determining the highest and lowest recovery rates among the 96 wells), and a standard deviation was calculated. For the SIO plate, the mean recovery was 95%, the range of recoveries was 11%, and the standard deviation was 2%. For the CA plate, the mean recovery was 64%, the range of recoveries was 14%, and the standard deviation was 3%.

The same test as in the preceding paragraph was also carried out using 96-well plates with 1000 μL wells. For the SIO plate, the mean recovery was 100%, the range of recoveries was 13%, and the standard deviation was 3%. For the CA plate, the mean recovery was 62%, the range of recoveries was 25%, and the standard deviation was 3%.

This testing indicated that the SIO treatment of Example 1 allows at least as uniform a recovery rate among the different wells as the protein resisting coating of the CA plate. This suggests that the SIO plasma treatment is very uniform across the plate.

### Second More Detailed Embodiment

A process according to a second more detailed embodiment has been developed that can be applied to polyolefins and a wide range of other polymers that optionally provides over 50% reduction in protein adhesion. The process is based on one to four steps or more that can take place at atmospheric and at reduced pressures via plasma processing. The process can be applied to a wide range of polymeric materials (polyolefins, polyesters, polystyrenes in addition to many other materials) and products including labware, diagnostic devices, contact lenses, medical devices, or implants in addition to many other products.

A first, optional step of the second more detailed embodiment is treating a surface with a polar liquid treatment agent comprising: water, a volatile, polar, organic compound, or a combination of any two or more of these, forming a polar-treated surface.

A second, optional step of the second more detailed embodiment is treating the surface with ionized gas.

A third, optional step of the second more detailed embodiment is treating the surface with conditioning plasma comprising: a nitrogen-containing gas, an inert gas, an oxidizing gas, or a combination of two or more of these, forming a conditioned surface.

A fourth step of the second more detailed embodiment is treating the surface with conversion plasma of water; a volatile, polar, organic compound; a C₁-C₁₂ hydrocarbon and oxygen; a C₁-C₁₂ hydrocarbon and nitrogen; a silicon-containing gas; or a combination of two or more of these, forming a converted surface.

The surface to be treated of the second more detailed embodiment can be made of a wide variety of different materials. Several useful types of materials are thermoplastic material, for example a thermoplastic resin, for example a polymer, optionally injection-molded thermoplastic resin. For example, the material can be, or include, an olefin polymer, polypropylene (PP), polyethylene (PE), cyclic olefin copolymer (COC), cyclic olefin polymer (COP), polymethylpentene, polyester, polyethylene terephthalate, polyethylene naphthalate, polybutylene terephthalate (PBT), polyvinylidene chloride (PVdC), polyvinyl chloride (PVC), polycarbonate, polylactic acid, polystyrene, hydrogenated polystyrene, polycyclohexylethylene (PCHE), epoxy resin, nylon, polyurethane polyacrylonitrile, polyacrylonitrile (PAN), an ionomeric resin, Surlyn® ionomeric resin, or any combination, composite or blend of any two or more of the above materials.

A wide variety of different surfaces can be treated according to the second more detailed embodiment. One example of a surface is a vessel lumen surface, where the vessel is, for example, a vial, a bottle, a jar, a syringe, a cartridge, a blister package, or an ampoule. For more examples, the surface of the material can be a fluid contact surface of an article of labware, for example a microplate, a centrifuge tube, a pipette tip, a well plate, a microwell plate, an ELISA plate, a microtiter plate, a 96-well plate, a 384-well plate, a centrifuge tube, a chromatography vial, an evacuated blood collection tube, or a specimen tube.

Yet another example of the second more detailed embodiment is that the surface can be a coating or layer of PECVD deposited SiOₓC_{y}H_{z} or SiNₓC_{y}H_{z}, in which x is from about 0.5 to about 2.4 as measured by X-ray photoelectron spectroscopy (XPS), y is from about 0.6 to about 3 as measured by XPS, and z is from about 2 to about 9 as measured by Rutherford backscattering spectrometry (RBS). Another example is the surface is a barrier coating or layer of SiOx, in which x is from about 1.5 to about 2.9 as measured by XPS, alternatively an oxide or nitride of an organometallic precursor that is a compound of a metal element from Group III and/or Group IV of the Periodic Table, e.g. in Group III: Boron, Aluminum, Gallium, Indium, Thallium, Scandium, Yttrium, or Lanthanum, (Aluminum and Boron being preferred), and in Group IV: Silicon, Germanium, Tin, Lead, Titanium, Zirconium, Hafnium, or Thorium (Silicon and Tin being preferred).

The polar liquid treatment agent of the second more detailed embodiment can be, for example, water, for example tap water, distilled water, or deionized water; an alcohol, for example a C₁-C₁₂ alcohol, methanol, ethanol, n-propanol, isopropanol, n-butanol, isobutanol, s-butanol, t-butanol; a glycol, for example ethylene glycol, propylene glycol, butylene glycol, polyethylene glycol, and others; glycerine, a C₁-C₁₂ linear or cyclic ether, for example dimethyl ether, diethyl ether, dipropyl ether, dibutyl ether, glyme (CH₃OCH₂CH₂OCH₃); cyclic ethers of formula —CH₂CH₂Oₙ— such as diethylene oxide, triethylene oxide, and tetraethylene oxide; cyclic amines; cyclic esters (lactones), for example acetolactone, propiolactone, butyrolactone, valerolactone, and caprolactone; a C₁-C₁₂ aldehyde, for example formaldehyde, acetaldehyde, propionaldehyde, or butyraldehyde; a C₁-C₁₂ ketone, for example acetone, diethylketone, dipropylketone, or dibutylketone; a C₁-C₁₂ carboxylic acid, for example formic acid, acetic acid, propionic acid, or butyric acid; ammonia, a C₁-C₁₂ amine, for example methylamine, dimethylamine, ethylamine, diethylamine, propylamine, butylamine, pentylamine, hexylamine, heptylamine, octylamine, nonylamine, decylamine, undecylamine, or dodecylamine; hydrogen fluoride, hydrogen chloride, a C₁-C₁₂ epoxide, for example ethylene oxide or propylene oxide; or a combination of any two or more of these. In this context, “liquid” means liquid under the temperature, pressure, or other conditions of treatment.

Contacting the surface with a polar liquid treatment agent of the second more detailed embodiment can be carried out in any useful manner, such as spraying, dipping, flooding, soaking, flowing, transferring with an applicator, condensing from vapor, or otherwise applying the polar liquid treatment agent. After contacting the surface with a polar liquid treatment agent of the second more detailed embodiment, the surface can be allowed to stand for 1 second to 30 minutes, for example.

In the ionized gas treatment of the second more detailed embodiment, the ionized gas can be, as some examples, air; nitrogen; oxygen; an inert gas, for example argon, helium, neon, xenon, or krypton; or a combination of any two or more of these. The ionized gas can be delivered in any suitable manner. For example, it can be delivered from an ionizing blow-off gun or other ionized gas source. A convenient gas delivery pressure is from 1-120 psi (6 to 830 kPa) (gauge or, alternatively, absolute pressure), optionally 50 psi (350 kPa). The water content of the ionized gas can be from 0 to 100%. The polar-treated surface with ionized gas can be carried out for any suitable treatment time, for example from 1-300 seconds, optionally for 10 seconds.

In the conditioning plasma treatment of the second more detailed embodiment, a nitrogen-containing gas, an inert gas, an oxidizing gas, or a combination of two or more of these can be used in the plasma treatment apparatus. The nitrogen-containing gas can be nitrogen, nitrous oxide, nitrogen dioxide, nitrogen tetroxide, ammonia, or a combination of any two or more of these. The inert gas can be argon, helium, neon, xenon, krypton, or a combination of any two or more of these. The oxidizing gas can be oxygen, ozone, or a combination of any two or more of these.

In the conversion plasma treatment of the second more detailed embodiment, water; a volatile, polar, organic compound; a C₁-C₁₂ hydrocarbon and oxygen; a C₁-C₁₂ hydrocarbon and nitrogen; a silicon-containing gas; or a combination of two or more of these can be used in the plasma treatment apparatus. The polar liquid treatment agent can be, for example, any of the polar liquid treatment agents mentioned in this specification. The C₁-C₁₂ hydrocarbon optionally can be methane, ethane, ethylene, acetylene, n-propane, i-propane, propene, propyne; n-butane, i-butane, t-butane, butane, 1-butyne, 2-butyne, or a combination of any two or more of these.

The silicon-containing gas of the second more detailed embodiment can be a silane, an organosilicon precursor, or a combination of any two or more of these. The silicon-containing gas can be an acyclic or cyclic, substituted or unsubstituted silane, optionally comprising, consisting essentially of, or consisting of any one or more of: Si₁-Si₄ substituted or unsubstituted silanes, for example silane, disilane, trisilane, or tetrasilane; hydrocarbon or halogen substituted Si₁-Si₄ silanes, for example tetramethylsilane (TetraMS), tetraethyl silane, tetrapropylsilane, tetrabutylsilane, trimethylsilane (TriMS), triethyl silane, tripropylsilane, tributylsilane, trimethoxysilane, a fluorinated silane such as hexafluorodisilane, a cyclic silane such as octamethylcyclotetrasilane or tetramethylcyclotetrasilane, or a combination of any two or more of these. The silicon-containing gas can be a linear siloxane, a monocyclic siloxane, a polycyclic siloxane, a polysilsesquioxane, an alkyl trimethoxysilane, a linear silazane, a monocyclic silazane, a polycyclic silazane, a polysilsesquiazane, or a combination of any two or more of these. The silicon-containing gas can be tetramethyldisilazane, hexamethyldisilazane, octamethyltrisilazane, octamethylcyclotetrasilazane, tetramethylcyclotetrasilazane, or a combination of any two or more of these.

The conditioning plasma treatment, the converting plasma treatment, or both of the second more detailed embodiment can be carried out in a plasma chamber. The plasma chamber can have a treatment volume between two metallic plates. The treatment volume can be, for example, from 100 mL to 50 liters, for example about 14 liters. Optionally, the treatment volume can be generally cylindrical.

The plasma chamber of the second more detailed embodiment can have a generally cylindrical outer electrode surrounding at least a portion of the treatment chamber.

To provide a gas feed to the plasma chamber of the second more detailed embodiment, a tubular gas inlet can project into the treatment volume, through which the feed gases are fed into the plasma chamber. The plasma chamber optionally can include a vacuum source for at least partially evacuating the treatment volume.

Optionally in the second more detailed embodiment, the exciting energy for the conditioning plasma or conversion plasma can be from 1 to 1000 Watts, optionally from 100 to 900 Watts, optionally from 500 to 700 Watts, optionally from 1 to 100 Watts, optionally from 1 to 30 Watts, optionally from 1 to 10 Watts, optionally from 1 to 5 Watts.

Optionally in the second more detailed embodiment, the plasma chamber is reduced to a base pressure from 0.001 milliTorr (mTorr) to 100 Torr before feeding gases in the conditioning plasma or conversion plasma treatment.

Optionally in the second more detailed embodiment, the gases are fed for conditioning plasma or conversion plasma treatment at a total pressure for all gases from 1 mTorr to 10 Torr, and at a feed rate of from 1 to 300 sccm, alternatively 1 to 100 sccm.

Optionally in the second more detailed embodiment, the gases are fed for conditioning plasma or conversion plasma treatment for from 1 to 300 seconds, alternatively from 90 to 180 seconds.

After the treatment(s) of the second more detailed embodiment, the treated surface, for example a vessel lumen surface, can be contacted with an aqueous protein. Some non-limiting examples of suitable proteins are the aqueous protein comprises: mammal serum albumin, for example Bovine Serum Albumin (BSA); Fibrinogen (FBG); Transferrin (TFN), for example blood serotransferrin (or siderophilin, also known as transferrin); lactotransferrin (lactoferrin); milk transferrin; egg white ovotransferrin (conalbumin); and membrane-associated melanotransferrin; Protein A (PrA); Protein G (PrG); Protein A/G; Protein L; Insulin, for example hexameric insulin, monomeric insulin, porcine insulin, human insulin, recombinant insulin and pharmaceutical grades of insulin; Pharmaceutical protein; blood or blood component proteins; or any recombinant form, modification, full length precursor, signal peptide, propeptide, or mature variant of these proteins; or a combination of two or more of these.

Optionally in the second more detailed embodiment, the converted surface has a protein recovery percentage greater than the protein recovery percentage of the untreated surface for at least one of Bovine Serum Albumin (BSA); Fibrinogen (FBG); Transferrin (TFN), for example blood serotransferrin (or siderophilin, also known as transferrin); lactotransferrin (lactoferrin); milk transferrin; egg white ovotransferrin (conalbumin); and membrane-associated melanotransferrin; Protein A (PrA); Protein G (PrG); Protein A/G; Protein L; Insulin, for example hexameric insulin, monomeric insulin, porcine insulin, human insulin, recombinant insulin and pharmaceutical grades of insulin; pharmaceutical protein; blood or blood component proteins; or any recombinant form, modification, full length precursor, signal peptide, propeptide, or mature variant of these proteins.

Optionally in the second more detailed embodiment, the converted surface has a protein recovery percentage at 24 hours greater than the protein recovery percentage of the untreated surface for Bovine Serum Albumin having an atomic mass of 66,000 Daltons (BSA) on NUNC® 96-well round bottom plates sold by Nunc A/S Corporation, Denmark, following the protocol in the present specification.

Optionally in the second more detailed embodiment, the converted surface has a protein recovery percentage at 24 hours on NUNC® 96-well round bottom plates greater than 70%, alternatively greater than 80%, alternatively greater than 90%, optionally up to 100% for BSA, following the protocol in the present specification.

Optionally in the second more detailed embodiment, the converted surface has a protein recovery percentage at 24 hours greater than the protein recovery percentage of the untreated surface for Fibrinogen having an atomic mass of 340,000 Daltons (FBG) on NUNC® 96-well round bottom plates, following the protocol in the present specification.

Optionally in the second more detailed embodiment, the converted surface has a protein recovery percentage at 24 hours on NUNC® 96-well round bottom plates greater than 20%, alternatively greater than 40%, alternatively greater than 60%, alternatively greater than 80%, optionally up to 84% for FBG, following the protocol in the present specification.

Optionally in the second more detailed embodiment, the converted surface has a protein recovery percentage at 24 hours on NUNC® 96-well round bottom plates greater than the protein recovery percentage of the untreated surface for Transferrin having an atomic mass of 80,000 Daltons (TFN), following the protocol in the present specification.

Optionally in the second more detailed embodiment, the converted surface has a protein recovery percentage at 24 hours on NUNC® 96-well round bottom plates greater than 60%, alternatively greater than 65%, alternatively greater than 69%, optionally up to 70% for TFN, following the protocol in the present specification.

Optionally in the second more detailed embodiment, the converted surface has a protein recovery percentage at 24 hours on NUNC® 96-well round bottom plates greater than the protein recovery percentage of the untreated surface for Protein A having an atomic mass of 45,000 Daltons (PrA), following the protocol in the present specification.

Optionally in the second more detailed embodiment, the converted surface has a protein recovery percentage at 24 hours on NUNC® 96-well round bottom plates greater than 9%, alternatively greater than 20%, alternatively greater than 40%, alternatively greater than 60%, optionally up to 67% for PrA, following the protocol in the present specification.

Optionally in the second more detailed embodiment, the converted surface has a protein recovery percentage at 24 hours on NUNC® 96-well round bottom plates greater than the protein recovery percentage of the untreated surface for Protein G having an atomic mass of 20,000 Daltons (PrG), following the protocol in the present specification.

Optionally in the second more detailed embodiment, the converted surface has a protein recovery percentage at 24 hours on NUNC® 96-well round bottom plates greater than 12%, alternatively greater than 20%, alternatively greater than 40%, alternatively greater than 60%, alternatively greater than 80%, alternatively up to 90% for PrG, following the protocol in the present specification.

Optionally in the second more detailed embodiment, the converted surface has a protein recovery percentage at 24 hours greater than the protein recovery percentage of the untreated surface for Bovine Serum Albumin having an atomic mass of 66,000 Daltons (BSA) on EPPENDORF® 96-well round bottom plates, following the protocol in the present specification. EPPENDORF® plates are sold by Eppendorf AG, Hamburg, Germany.

Optionally in the second more detailed embodiment, the converted surface has a protein recovery percentage at 24 hours on EPPENDORF® 96-well round bottom plates greater than 95% for BSA, following the protocol in the present specification.

Optionally in the second more detailed embodiment, the converted surface has a protein recovery percentage at 24 hours greater than the protein recovery percentage of the untreated surface for Fibrinogen having an atomic mass of 340,000 Daltons (FBG) on EPPENDORF® 96-well round bottom plates, following the protocol in the present specification.

Optionally in the second more detailed embodiment, the converted surface has a protein recovery percentage at 24 hours on EPPENDORF® 96-well round bottom plates greater than 72% for FBG, following the protocol in the present specification.

Optionally in the second more detailed embodiment, the converted surface has a protein recovery percentage at 24 hours on EPPENDORF® 96-well round bottom plates greater than the protein recovery percentage of the untreated surface for Transferrin having an atomic mass of 80,000 Daltons (TFN), following the protocol in the present specification.

Optionally in the second more detailed embodiment, the converted surface has a protein recovery percentage at 24 hours on EPPENDORF® 96-well round bottom plates greater than 69% for TFN, following the protocol in the present specification.

Optionally in the second more detailed embodiment, the converted surface has a protein recovery percentage at 24 hours on EPPENDORF® 96-well round bottom plates greater than the protein recovery percentage of the untreated surface for Protein A having an atomic mass of 45,000 Daltons (PrA), following the protocol in the present specification.

Optionally in the second more detailed embodiment, the converted surface has a protein recovery percentage at 24 hours on EPPENDORF® 96-well round bottom plates greater than the protein recovery percentage of the untreated surface for Protein G having an atomic mass of 20,000 Daltons (PrG), following the protocol in the present specification.

Optionally in the second more detailed embodiment, the converted surface has a protein recovery percentage at 24 hours on EPPENDORF® 96-well round bottom plates greater than 96% for PrG, following the protocol in the present specification.

Optionally in the second more detailed embodiment, the converted surface has a protein recovery percentage at 24 hours greater than the protein recovery percentage of the untreated surface for Bovine Serum Albumin having an atomic mass of 66,000 Daltons (BSA) on GRIENER® 96-well round bottom plates, following the protocol in the present specification. GRIENER® plates are sold by Greiner Holding AG of Austria.

Optionally in the second more detailed embodiment, the converted surface has a protein recovery percentage at 24 hours on GRIENER® 96-well round bottom plates greater than 60%, alternatively up to 86%, for BSA, following the protocol in the present specification.

Optionally in the second more detailed embodiment, the converted surface has a protein recovery percentage at 24 hours greater than the protein recovery percentage of the untreated surface for Fibrinogen having an atomic mass of 340,000 Daltons (FBG) on GRIENER® 96-well round bottom plates, following the protocol in the present specification.

Optionally in the second more detailed embodiment, the converted surface has a protein recovery percentage at 24 hours on GRIENER® 96-well round bottom plates greater than 50%, alternatively up to 65%, for FBG, following the protocol in the present specification.

Optionally in the second more detailed embodiment, the converted surface has a protein recovery percentage at 24 hours on GRIENER® 96-well round bottom plates greater than the protein recovery percentage of the untreated surface for Transferrin having an atomic mass of 80,000 Daltons (TFN), following the protocol in the present specification.

Optionally in the second more detailed embodiment, the converted surface has a protein recovery percentage at 24 hours on GRIENER® 96-well round bottom plates greater than 50%, alternatively up to 60%, for TFN, following the protocol in the present specification.

Optionally in the second more detailed embodiment, the converted surface has a protein recovery percentage at 24 hours on GRIENER® 96-well round bottom plates greater than the protein recovery percentage of the untreated surface for Protein A having an atomic mass of 45,000 Daltons (PrA), following the protocol in the present specification.

Optionally in the second more detailed embodiment, the converted surface has a protein recovery percentage at 24 hours on GRIENER® 96-well round bottom plates greater than 25%, alternatively up to 56%, for PrA, following the protocol in the present specification.

Optionally in the second more detailed embodiment, the converted surface has a protein recovery percentage at 24 hours on GRIENER® 96-well round bottom plates greater than the protein recovery percentage of the untreated surface for Protein G having an atomic mass of 20,000 Daltons (PrG), following the protocol in the present specification.

Optionally in the second more detailed embodiment, the converted surface has a protein recovery percentage at 24 hours on GRIENER® 96-well round bottom plates greater than 60%, alternatively up to 75%, for PrG, following the protocol in the present specification.

### Working Example 14

The following is a description and working example of the process of the second more detailed embodiment:

The process of the second more detailed embodiment was applied to 96-well polypropylene microplates manufactured by NUNC®.

The following steps of the second more detailed embodiment were applied to the parts:

As received plates were contacted according to the second more detailed embodiment by being sprayed with tap water (de-ionized or other waters could be used, as could any polar solvent), referred to here as a polar liquid treatment agent, and allowed to stand for I second to 30 minutes, providing a polar-treated surface.

The parts were then blown off with ionized air according to the second more detailed embodiment, which is referred to here as contacting the polar-treated surface with ionized gas at a pressure of 50 psi. Alternatively, a gas (nitrogen, argon or any other compressed gas) could be used in place or in addition to the air. The water content of the gas (being used to blow off the parts) can be 0-100%. The parts were blown off for approximately 10 seconds although a time from 1-300 seconds could be used.

The parts were then loaded onto a carrier for the next step of the second more detailed embodiment. A holding time from 1-300 seconds prior to loading or once loaded (For a total of 1-600 seconds) can be used.

The parts were then loaded into a plasma chamber for treating the ionized-pressurized-gas-treated surface with conditioning plasma according to the second more detailed embodiment. It is theorized, without limiting the invention according to the scope or accuracy of this theory, that the conditioning plasma of the second more detailed embodiment cleans non-polymer additives from the surface of the microplates and/or creates a hydrophilic, nanotextured surface, also known as a nanostructure of peaks and recesses, amenable to surface functionalization. According to this theory, the nanostructure would facilitate hydrophilization of the “peaks” while sterically preventing comparatively large proteins from accessing any hydrophobic recesses. Further according to this theory, plasma conditioning, also known as activation, might be better accomplished utilizing an amine (radical) function during the conditioning step, which can be a “handle” or attachment point further built upon or modified in the conversion step, versus a hydroxyl (radical) function or methyl/methylene radicals, when considering the relative stability of the radicals generated (an amine radical is more stable, for example, than a hydroxyl radical, and easier to form than a methyl radical).

An exemplary plasma treatment chamber of the second more detailed embodiment, used in the present example, had the configuration shown in FIG. 10. (alternative chambers can be used as well—see below):

Referring to FIG. 10 of the second more detailed embodiment, a cylindrical ceramic chamber 110 is shown, with an aluminum bottom 112 and an aluminum lid 114 (which was closed during use, but shown open in FIG. 10, as it would be when loading or unloading). The chamber 110 was approximately 12 inches (30 cm) in diameter and 8 inches (20 cm) deep. The pumping port of the chamber feeding the vacuum conduit 116 to the vacuum pump 118, controlled by a valve 20, was at the bottom (in the aluminum bottom 112) and was approximately 4 inches (10 cm) in diameter, with the ½-inch (12 mm) diameter gas inlet 122 concentrically protruding through the pumping port into the processing area 124. A plasma screen (not shown) was installed in over the pumping port and was constructed from copper screen and steel wool. Gas was fed to the gas inlet 122 via a gas system 126 under the chamber 10. Mass flow controllers such as 128 were used for the compressed gas (e.g. from the source 130) and a capillary 132 (0.006 inches (0.15 mm) internal ID), that was 36 inches (90 cm) long, controlled the feed rate of water into the manifold 134, via a shut-off valve 136. The ceramic chamber 110 had a copper electrode 138 that was concentrically wrapped around the outside and was approximately 7 inches (18 cm) tall. The electrode 138 was connected to a COMDEL® matching network 140 that allowed the 50-ohm output of the COMDEL® 1000-watt RF (13.56 MHz) power supply 42 to be matched for optimal power coupling (low reflected power). COMDEL® equipment is sold by Comdel, Inc., Gloucester, Mass., USA. The power supply 142 was attached to the COMDEL® matching network 40 via a standard coaxial cable 144. Two capacitance manometers (0-1 Torr and 0-100 Torr) (not shown) were attached to the vacuum conduit 116 (also referred to as a pump line) to measure the process pressures.

The process of the second more detailed embodiment can occur in a wide range of plasma processing chambers including through the use of atmospheric plasma(s) or jets. The parts can be processed in batch (as described above) of 1-1000 parts or processed in a semi-continuous operation with load-locks. In the case of atmospheric processing, no chamber would be required. Alternatively, single parts can be processed as described in FIG. 2 and the accompanying description in U.S. Pat. No. 7,985,188.

Once loaded for treating the ionized-pressurized-gas-treated surface with conditioning plasma of the second more detailed embodiment, the pressure inside of the chamber was reduced to 50 mTorr. Base pressures to 10⁻⁶ Torr or as high as 100 Torr are also acceptable. Once the base pressure was reached, nitrogen gas (99.9% pure, although purities as high as 99.999% or as low as 95% can also be used) at 30 sccm (standard cubic centimeters per minute) was admitted to the chamber, achieving a processing pressure of 40 mTorr (pressures as low as 1 mTorr or as high as 10 Torr can also be used). A plasma was then ignited using 600 watts at a frequency of 13.56 MHz for 90-180 seconds, although processing times from 1-300 seconds will work. Frequencies from 1 Hz to 10 GHz are also possible. After the processing time was complete the plasma was turned off and the gas evacuated (although this is not a requirement) back to the base pressure. This conditioning plasma treatment of the second more detailed embodiment produced a conditioned surface on the microplates.

Next, the conditioned surface was treated with conversion plasma of the second more detailed embodiment, in the same apparatus, although other apparatus may instead be used.

It is theorized as one mechanism of action of the second more detailed embodiment, without limiting the invention according to the scope or accuracy of this theory, that the conversion plasma provides further functionalization (hydrophilic “extenders”) of the hydrophilized sites or “handles” resulting from the conditioning plasma treatment. According to this theory, this extension function may be accomplished by established (1) polyethylene oxide condensation (PEGylation) methods or (2) betaine/zwitterion (internal ion pair) methods via plasma activation of the surface “handle” (functional group from the conditioning plasma treatment) and/or the hydrophilic extender species. According to this theory, the “extenders” referred to here are the components of the conversion plasma. According to this theory, the recombination of the surface “handle” and “extender” species to form larger hydrophilic species on the treated surface provides a hydrophilic sterically stabilized surface, providing inhibiting protein surface binding via hydrosteric repulsion. According to this theory, cyclic esters (lactones) or cyclic sulfoesters (sultones) could be utilized to generate betaine or sulfobetaine species on the treated surface.

The conversion plasma was applied as follows according to the second more detailed embodiment. The chamber was evacuated (or remained evacuated), and water vapor was flowed into the chamber through a 0.006 inch (0.15 mm) diameter capillary (36 inches (91 cm) long) at an approximate flow of 30 sccm resulting in a processing pressure between 26 and 70 mTorr (milliTorr). The flow of water vapor can range from 1-100 sccm and pressures from 1 mTorr to 100 Torr are also possible. A plasma was then ignited at 600 watts and sustained for 90-180 seconds although processing times from 1-300 seconds will work. The plasma was then turned off, the vacuum pump valves closed and then the chamber vented back to atmosphere. A converted surface was formed as a result. Room air was used to vent the chamber although nitrogen could be used. Alternatively, water vapor or other polar solvent containing material could be used.

Once the chamber of the second more detailed embodiment was vented, the lid was removed and the carrier removed. The parts were then unloaded. The parts are ready to use at that point, or they can be packaged in plastic bags, aluminum foil or other packaging for storage and shipment.

The resulting surface (from the above treatment of the second more detailed embodiment) provided a significant reduction in protein adhesion. The reduction is believed to be due to a high hydroxyl content on the surface. The results are shown in Tables 13-16.

Similar processing of the second more detailed embodiment can be used to process a wide variety of other articles. These include: labware, for example a fluid contact surface of a microplate, a centrifuge tube, a pipette tip, a well plate, a microwell plate, an ELISA plate, a microtiter plate, the illustrated 96-well plate, a 384-well plate; vessels, for example a vial, a bottle, a jar, a syringe, a cartridge, a blister package, an ampoule, an evacuated blood collection tube, a specimen tube, a centrifuge tube, or a chromatography vial; or medical devices having surfaces that come in contact with blood and other body fluids or pharmaceutical preparations containing proteins, such as catheters, stents, heart valve, electrical leads, pacemakers, insulin pumps, surgical supplies, heart-lung machines, contact lenses, etc.

### Alternative Processes of the Second More Detailed Embodiment

Water can be applied to the part (via a mist or high humidity cabinet of the second more detailed embodiment) as described above then:
Blowing part/product off with ionized air as described above of the second more detailed embodiment then:
A pre-treatment of the second more detailed embodiment at reduced pressure utilizing a plasma (ionized gas) comprising Nitrogen, then a final treatment of one of the following:
   i. Methane and air
   ii. Methane and nitrogen
   iii. Methane and water
   iv. Any combination of the above
   v. Any other hydrocarbon gas
   vi. Silane and nitrogen
   vii. Silane and water
   viii. Any organosilicon in place of the silane

TABLE 13
of the second more detailed embodiment:
Results
(See notes at the end of this section for definitions of each column)
RECOVERY @ 24 hrs in %

| Treatment | Plate | BSA | FBG | TFN | PrA | PrG |
|---|---|---|---|---|---|---|
| U/C | Nunc | 0 | 6 | 55 | 3 | 4 |
| N | Nunc | 73 | 31 | 51 | 9 | 12 |
| H | Nunc | 78 | 21 | 54 | 22 | 25 |
| I/+/H | Nunc | 82 | 42 | 60 | 78 | 89 |
| U/C | Epp | 93 | 62 | 68 | 86 | 96 |
| N | Epp | 107 | 72 | 67 | 82 | 103 |
| Lipidure | Nunc | 71 | 76 | 65 | 80 | 88 |
| ns3 12/14 | Nunc | 101 | 84 | 70 | 64 | 85 |

TABLE 14
of the second more detailed embodiment
NUNC 96 well round bottom plates

| Condition | Spray | W/D | Ionize | N time | H time | Power | BSA | FBG | PrA | PrG | TFN |
|---|---|---|---|---|---|---|---|---|---|---|---|
| 5 | Y | W | Y | 90 | 180 | Std | 99 | 84 | 67 | 90 | 55 |
| 2 | N | | Y | 180 | 180 | Std | 101 | 84 | 84 | 85 | 70 |
| 4 | Y | D | Y | 90 | 180 | Std | 103 | 77 | 65 | 79 | 68 |
| 3 | Y | D | Y | 180 | 180 | Std | 91 | 74 | 61 | 76 | 65 |
| 7 | Y | D | Y | 90 | 180 | 50% | 90 | 69 | 57 | 73 | 62 |
| 6 | N | | Y | 90 | 180 | 50% | 89 | 65 | 56 | 73 | 65 |
| 1 | Y | W | Y | 180 | 180 | Std | 95 | 58 | 54 | 72 | 65 |

TABLE 15
of the second more detailed embodiment

| | | | | | Griener 96 well round bottom plates | | | | | | |
|---|---|---|---|---|---|---|---|---|---|---|---|
| Condition | Spray | W/D | Ionize | N time | H time | Power | BSA | FBG | PrA | PrG | TFN |
| 4 | Y | D | Y | 90 | 180 | Std | 86 | 54 | 56 | 69 | 57 |
| 6 | N | | Y | 90 | 180 | 50% | 81 | 56 | 48 | 75 | 60 |
| 3 | Y | D | Y | 180 | 180 | Std | 76 | 64 | 38 | 73 | 57 |
| 5 | Y | W | Y | 90 | 180 | Std | 86 | 65 | 34 | 61 | 58 |
| 7 | Y | D | Y | 90 | 180 | 50% | 83 | 54 | 35 | 64 | 56 |
| 1 | Y | W | Y | 180 | 180 | Std | 77 | 65 | 27 | 63 | 54 |
| 2 | N | | Y | 180 | 180 | Std | 71 | 57 | 34 | 68 | 55 |

TABLE 16
of the second more detailed embodiment

| | | | | | vs. Sample 1, | | |
|---|---|---|---|---|---|---|---|
| | | | | | same base material | | |
| | | | | Conversion | XPS: | SIMS: | |
| | Base | SiOₓ | SiOₓC_{y}H_{z} | Plasma | —OH | —OH | Contact |
| SAMPLE | Material | ? | ? | Treatment? | content | content | Angle |
| 1 | COC | no | no | no | = | = | = |
| 1 | COP | no | no | no | = | = | = |
| 2 | COC | yes | yes | no | na | na | > |
| 2 | COP | yes | yes | no | na | na | > |
| 3 | COC | yes | yes | yes | > | > | < |
| 3 | COP | yes | yes | yes | > | > | < |
| 4 | COC | no | no | yes | > | > | < |
| 4 | COP | no | no | yes | > | > | < |

### NOTES to Tables of the Second More Detailed Embodiment:

Treatment—This indicates if the plates were treated with the process of the second more detailed embodiment described herein (ns3, N—nitrogen plasma only (treating the ionized-pressurized-gas-treated surface with conditioning plasma), H—water plasma only (treating the conditioned surface with conversion plasma comprising: water, a volatile, polar, organic compound, a C₁-C₁₂ hydrocarbon and oxygen, a hydrocarbon and nitrogen, a silicon-containing gas, or a combination of two or more of these, forming a converted surface), 1/+/H—ionize, nitrogen plasma and water plasma, i.e. contacting the polar-treated surface with ionized gas; treating the ionized-pressurized-gas-treated surface with conditioning plasma, forming a conditioned surface; and treating the conditioned surface with conversion plasma), U/C—uncoated or treated, these were the as-received plates, Lipidure—this is a commercially available liquid applied and cured chemistry Plate—NUNC®—Epp is short for EPPENDORF® a plastic manufacturer

Spray—indicates plates were “misted” or sprayed with water prior to coating of the second more detailed embodiment. This was an example of contacting the surface with a polar liquid treatment agent comprising: water, a volatile, polar, organic compound, or a combination of any two or more of these, forming a polar-treated surface.

W/D indicates if the plates were sprayed and then immediately blown off with ionized air (W) or if they were left for 1-20 minutes and then blown off with ionized air (D) (contacting the polar-treated surface with ionized gas), in either event of the second more detailed embodiment.

N—time=nitrogen gas treatment time in seconds.
H—time=water gas treatment time in seconds
Power—Std was 600 watts applied RF power, 50% was 300 wafts.
BSA, FBG, PrA, PrG, TFN were all the proteins used in the study.

### Testing for Hydroxyl Content of the Second More Detailed Embodiment

The converted surface made by each described process, for example a 96-well microplate well surface, is analyzed by XPS, SIMS, and contact angle with water, compared to the untreated surface, to determine differences resulting from the present treatments. The following results are contemplated.

### XPS Analysis of the Second More Detailed Embodiment

X-ray photoelectron spectroscopy (XPS) is used to characterize the atomic composition of the treated surface of the second more detailed embodiment, before any or all of the presently contemplated polar liquid, ionized gas, conditioning plasma, or conversion plasma treatments, versus after the conversion plasma treatment of the second more detailed embodiment.

XPS data of the second more detailed embodiment is quantified using relative sensitivity factors and a model that assumes a surface having a different composition than the bulk material just below the surface, since the present treatments are believed to modify underlying structure at the surface rather than coating it or modifying the bulk material. The analysis volume is the product of the analysis area (spot size or aperture size) and the depth of information. Photoelectrons are generated within the X-ray penetration depth (typically many microns), but only the photoelectrons within the top escape depth or depths are detected.

A suitable XPS instrument of the second more detailed embodiment is a PHI™ Quantum 2000 XPS system, sold by Physical Electronics, Inc., Eden Prairie, Minn., USA. The PHI™ XPS uses as the X-ray source Monochromated Alka 1486.6 eV x-ray energy. Suitable Acceptance Angle, Take-Off Angle, Analysis Area, Charge Correction, Ion Gun, and Sputter Rate conditions are used during analysis. Values given are normalized to 100 percent using the elements detected. Detection limits are approximately 0.05 to 1.0 atomic percent.

XPS does not measure or detect hydrogen, but does detect and measure oxygen, silicon, nitrogen, and carbon. Consequently, when using XPS in search of an increase in surface hydroxyl concentration, the artifact searched for is an increase in oxygen at the surface of the article, compared to the oxygen level of the untreated surface and/or the oxygen level beneath the treated surface.

XPS analysis is used of the second more detailed embodiment to determine whether treatments using any of the present polar liquid treatment agent, ionized gas, conditioning plasma, and/or conversion plasma treatment steps, or the cumulative result of these treatments, causes an increase in oxygen concentration at the treated surface. It is contemplated that, at the conclusion of the conversion plasma treatment, the level of oxygen detected at the treated surface is greater than the level of oxygen beneath the treated surface, and also greater than the level of oxygen detected at a corresponding surface before any treatment with a polar liquid, ionized gas, conditioning plasma, or conversion plasma.

### SIMS Analysis

Secondary-ion mass spectrometry (SIMS) of the present treated and untreated samples is contemplated of the second more detailed embodiment. SIMS is used to analyze the composition of solid surfaces and thin films. In SIMS, the treated and untreated surfaces are addressed by directing a focused ion beam, which may have various compositions, against each surface, which ejects secondary ions from the surface materials. The mass/charge ratios of the secondary ions are measured with a mass spectrometer to determine the chemical composition of the surface to a depth of 1 to 2 nm (nanometers). Since oxygen and hydroxyl groups are being sought, it is contemplated that the ion beam will not be an oxygen beam, which would interfere with detection. The ion beam can, for example, be a cesium ion beam which is readily distinguishable from all constituents of the untreated and treated surface and the bulk sample. It is contemplated that the result of SIMS analysis of the second more detailed embodiment will be an increase in the total of oxygen, hydroxyl, and oxygen-containing moieties detected from the converted surface, versus the untreated surface.

### Contact Angle Analysis

A contact angle analysis can be done on the treated surface versus an untreated surface of the second more detailed embodiment, to determine the effect of the present treatments.

Three kinds of vials of the second more detailed embodiment are analyzed for the contact angle with distilled water on the interior surface. The specimens are (A) an untreated cyclic olefin polymer (COP) vial, (B) a similar vial having an SiOₓ oxygen and solute barrier plus an SiOₓC_{y}H_{z} pH protective coating on its inner surface, as described for example in U.S. Publ. Appl. 2015/0021339, published Jan. 22, 2015, and (C) a glass vial. Small pieces are obtained by cutting the plastic vials or crushing the glass vial in order to test the inside surface.

The analysis instrument for the contact angle tests of the second more detailed embodiment is the Contact Angle Meter model DM-701, made by Kyowa Interface Science Co., Ltd. (Tokyo, Japan). To obtain the contact angle, five water droplets are deposited on the inside surface of small pieces obtained from each specimen. The testing conditions and parameters are summarized below. Both plastic vials are cut and trimmed, while the glass vial needs to be crushed. The best representative pieces for each specimen are selected for testing. A drop size of 1 μL (one microliter) is used for all samples. Due to the curvature of the specimens, a curvature correction routine is used to accurately measure the contact angle.

### Contact Angle Testing Conditions and Parameters of the Second More Detailed Embodiment

Test instrument—DM-701 Contact Angle Meter
Liquid Dispenser—22 gauge stainless steel needle
Drop Size—μL (one microliter)
Test liquid—Distilled water
Environment—Ambient air, room temperature (about 25° C.)
Radius of Curvature for each Vial Specimen (micrometers, μm)—
   COP=9240 μm
   COP plus barrier and pH protective coatings or layers=9235 μm
   Glass=9900 μm

### Results of the Second More Detailed Embodiment

The specimen made from COP plus SiOₓC_{y}H_{z} pH protective coating of the second more detailed embodiment as described in this specification has the highest average contact angle of all tested specimens. The average contact angle for specimens made from COP plus pH protective coating is 99.1° (degrees). The average contact angle for the uncoated COP specimen is 90.5°. The glass specimen has a significantly lower average contact angle at 10.6°. This data shows that the pH protective coating raises the contact angle of the uncoated COP vessel. It is expected that an SiOₓ coated vessel without the passivation layer or pH protective coating would exhibit a result similar to glass, which shows a hydrophilic coating relative to the relative to the passivation layer or pH protective coating.

The present conversion plasma treatment of the second more detailed embodiment is expected to lower the contact angle of the substrate substantially, whether applied directly to the COP substrate or to an SiOₓC_{y}H_{z} (pH protective coating or layer) surface. Contact angles of 5° to 90°, alternatively 10° to 70°, alternatively 10° to 40° are contemplated to be achievable using the present conversion plasma treatment, optionally with other treatments, on a surface.

### Example—Vials with pH Protective Coating or Layer of the Second More Detailed Embodiment

A cyclic olefin copolymer (COC) resin is injection molded to form a batch of 5 ml COC vials. A cyclic olefin polymer (COP) resin is injection molded to form a batch of 5 ml COP vials. XPS, SIMS, and contact angle tests are performed on the COC and COP vials, as made. These vials are referred to below as Sample 1 vials.

Samples of the respective COC and COP vials are coated by identical processes, of the second more detailed embodiment as described in this example. The COP and COC vials are coated with a two layer coating by plasma enhanced chemical vapor deposition (PECVD). The first layer is composed of SiOₓ with oxygen and solute barrier properties, and the second layer is an SiOₓC_{y}H_{z} pH protective coating or layer. (Alternatively, other deposition processes than PECVD (plasma-enhanced chemical vapor deposition), such as non-plasma CVD (chemical vapor deposition), physical vapor deposition (in which a vapor is condensed on a surface without changing its chemical constitution), sputtering, atmospheric pressure deposition, and the like can be used, without limitation).

To form the SiOₓC_{y}H_{z} pH protective coating or layer of the second more detailed embodiment, a precursor gas mixture comprising OMCTS, argon, and oxygen is introduced inside each vial. The gas inside the vial is excited between capacitively coupled electrodes by a radio-frequency (13.56 MHz) power source. The preparation of these COC vials and the corresponding preparation of these COP vials, is further described in Example DD and related disclosure of US Publ. Appl. 2015-0021339 A1. XPS, SIMS, and contact angle tests are performed on the resulting SiOₓC_{y}H_{z} pH protective coating or layer surfaces of the COC and COP vials. These vials are referred to below as Sample 2 vials.

The interiors of the COC and COP vials are then further treated with conditioning plasma of the second more detailed embodiment, using nitrogen gas as the sole feed, followed by conversion plasma of the second more detailed embodiment, using water vapor as the sole feed, both as described in this specification, to provide vials having converted interior surfaces. XPS, SIMS, and contact angle tests are performed on the resulting converted interior surfaces of the COC and COP vials. These vials are referred to below as Sample 3 vials.

Vials identical to the Sample 1 vials, without SiOₓ or SiOₓC_{y}H_{z} coatings, are also directly treated with conditioning plasma of the second more detailed embodiment, using nitrogen gas as the sole feed, followed by a conversion plasma of the second more detailed embodiment a, using water vapor as the sole feed, both as described in this specification, to provide vials having converted interior surfaces. These vials are referred to below as Sample 4 vials.

The respective vials and expected test results of the second more detailed embodiment are summarized in Table 16 of the second more detailed embodiment. In Table 16 of the second more detailed embodiment, “=” means equal to Sample 1 on the same substrate (COC or COP), “<” means less than Sample 1 on the same substrate, and “>” means greater than Sample 1 on the same substrate.

While the invention has been described in detail and with reference to specific examples and embodiments thereof, it will be apparent to one skilled in the art that various changes and modifications can be made therein without departing from the spirit and scope thereof. Additional disclosure is provided in the claims, which are considered to be a part of the present description, each claim defining an alternative and optional embodiment.

What is claimed is:

1. A method for treating a surface, optionally a surface of a substrate or a surface of a material, comprising: treating the surface with conditioning plasma, conversion plasma, or both of one or more non-polymerizing compounds to form a converted surface.

2. The method of claim 1, in which treating the surface is carried out by:
employing a first treatment step that includes remote conditioning treatment of the surface with conditioning plasma of one or more non-polymerizing compounds at a remote point, where the ratio of the radiant energy density at the remote point to the radiant energy density at the brightest point of the conditioning plasma is less than 0.5, alternatively less than 0.25, alternatively substantially zero, alternatively zero; and
employing a second treatment step that includes remote conversion treatment of the surface with a conversion plasma of water vapor to form a converted surface, where the ratio of the radiant energy density at the remote point of conversion treatment to the radiant energy density at the brightest point of the conversion plasma is less than 0.5, alternatively less than 0.25, alternatively substantially zero, alternatively zero;
wherein after the second treatment step the converted surface has a biomolecule recovery percentage greater than the biomolecule recovery percentage of the surface prior to treatment according to the method, optionally after 24 hours.

3. The method of claim 1, wherein the treated surface has a biomolecule recovery percentage of at least 40%, optionally at least 45%, optionally at least 50%, optionally at least 55%, optionally at least 60%, optionally at least 65%, optionally at least 70%, optionally at least 75%, optionally at least 80%, optionally at least 85%, optionally at least 90% optionally at least 95%, wherein the biomolecule recovery percentage exceeds the biomolecule recovery percentage of the surface prior to treatment according to the method.

4. The method of claim 1, wherein the biomolecule recovery percentage of the treated surface is at least 80%, optionally at least 82%, optionally at least 86%.

5. The method of claim 1, wherein the biomolecule recovery percentage of the treated surface is from 82% to 90%, optionally about 86%, or about 88% or about 90%.

6. The method of claim 1, wherein the surface is a vessel lumen surface.

7. The method of claim 1, wherein the converted surface has a biomolecule recovery percentage greater than the biomolecule recovery percentage of the untreated surface for at least one of: mammal serum albumin; Bovine Serum Albumin (BSA); Fibrinogen (FBG); Transferrin (TFN); egg white ovotransferrin (conalbumin); membrane-associated melanotransferrin; Protein A (PrA); Protein G (PrG); Protein A/G; Protein L; Insulin; Pharmaceutical protein; blood or blood component proteins; and any recombinant form, modification, full length precursor, signal peptide, propeptide, or mature variant of these proteins.

8. The method of claim 1, wherein the substrate comprises thermoplastic material, for example a thermoplastic resin, for example an injection-molded thermoplastic resin.

9. The method of claim 1, wherein the substrate comprises olefin polymer, polypropylene (PP), polyethylene (PE), cyclic olefin copolymer (COC), cyclic olefin polymer (COP), polymethylpentene, polyester, polyethylene terephthalate, polyethylene naphthalate, polybutylene terephthalate (PBT), polyvinylidene chloride (PVdC), polyvinyl chloride (PVC), polycarbonate, polylactic acid, polystyrene, hydrogenated polystyrene, polycyclohexylethylene (PCHE), epoxy resin, nylon, polyurethane polyacrylonitrile, polyacrylonitrile (PAN), an ionomeric resin, Surlyn® ionomeric resin, or any combination, composite or blend of any two or more of the above materials.

10. The method of claim 1 wherein the first treatment step and/or second treatment step are carried out using plasma excited by extremely low frequency (ELF) of 3 to 30 Hz, super low frequency (SLF) of 30 to 300 Hz, voice or ultra-low frequency (VF or ULF) of 300 Hz to 3 kHz, very low frequency (VLF) of 3 to 30 kHz, low frequency (LF) of 30 to 300 kHz, medium frequency (MF) of 300 kHz to 3 MHz, high frequency (HF) of 3 to 30 MHz, very high frequency (VHF) of 30 to 300 MHz, ultra-high frequency (UHF) of 300 MHz to 3 GHz, or any combination of two or more of these.

11. The method of claim 1, in which the surface is a coating or layer of PECVD deposited SiOₓC_{y}H_{z} or SiNₓC_{y}H_{z}, in which x is from about 0.5 to about 2.4 as measured by X-ray photoelectron spectroscopy (XPS), y is from about 0.6 to about 3 as measured by XPS, and z is from about 2 to about 9 as measured by Rutherford backscattering spectrometry (RBS); or a barrier coating or layer of SiOₓ, in which x is from about 1.5 to about 2.9 as measured by XPS, alternatively an oxide or nitride of an organometallic precursor that is a compound of a metal element from Group III and/or Group IV of the Periodic Table, e.g. in Group III: Boron, Aluminum, Gallium, Indium, Thallium, Scandium, Yttrium, or Lanthanum, (Aluminum and Boron being preferred), and in Group IV: Silicon, Germanium, Tin, Lead, Titanium, Zirconium, Hafnium, or Thorium (Silicon and Tin being preferred).

12. (canceled)

13. The method of claim 1, wherein the surface of the substrate is a fluid contact surface of an article of labware.

14. The method of claim 1, wherein the surface of the substrate is a fluid contact surface of a microplate, a centrifuge tube, a pipette tip, a well plate, a microwell plate, an ELISA plate, a microtiter plate, a 96-well plate, a 384-well plate, a vial, a bottle, a jar, a syringe, a cartridge, a blister package, an ampoule, an evacuated blood collection tube, a specimen tube, a centrifuge tube, or a chromatography vial.

15. The method of claim 1, wherein the method is carried out in a plasma chamber having a treatment volume of 100 mL to 50 liters, for example about 8 to 20 liters, wherein the treatment volume is optionally generally cylindrical; and optionally the plasma chamber further comprises a generally cylindrical outer applicator or electrode surrounding at least a portion of the treatment chamber and optionally a tubular fluid inlet projects into the treatment volume, through which the feed gases are fed into the plasma chamber; and optionally the plasma chamber further comprises a vacuum source for at least partially evacuating the treatment volume.

16.-108. (canceled)

109. A method for treating a surface and or coating on a surface of a material that improves protein recovery rates comprising the steps of:
applying a solvent, also known as a polar liquid treatment agent, to the surface, and
applying ionized gas to the surface, and
creating a first gas plasma, also known as conditioning plasma, at the surface, and
creating a second gas plasma, also known as conversion plasma, at the surface.

110. (canceled)

111. The method of claim 109 where the ionized gas is air.

112. The method of claim 109 where the first gas plasma, also known as conditioning plasma, is comprised of nitrogen.

113. The method of claim 109 where the second gas plasma, also known as conversion plasma, is comprised of water.

114.-117. (canceled)

118. A method for creating a surface and or coating on a surface of a material that improves protein recovery rates comprising the steps of:
applying a solvent, also known as a polar liquid treatment agent, to the surface, and
creating a first gas plasma, also known as conditioning plasma at the surface, and
creating a second gas plasma, also known as conversion plasma, of a solvent at the surface

119. The method of claim 118 where the first solvent, also known as a polar liquid treatment agent is water.

120. The method of claim 118 where the first gas plasma, also known as conditioning plasma, is comprised of nitrogen.

121. The method of claim 118 where the second gas plasma, also known as conversion plasma, is comprised of water.

122.-154. (canceled)

155. An article as described in claim 1, treated according to a method as described in claim 1.
